(19) United States
(12) Patent Application Publication
(10) Pub. No.: US 2025/0362286 A1
(43) Pub. Date: Nov. 27, 2025

(54) SCREENING MODELS AND METHODS OF CANCER TREATMENT

(71) Applicant: The Regents of the University of California, Oakland, CA (US)

(72) Inventors: Stephanie Fraley, La Jolla, CA (US); Sural Ranamukhaarachchi, La Jolla, CA (US)

(21) Appl. No.: 18/872,716

(22) PCT Filed: Jun. 8, 2023
(86) PCT No.: PCT/US2023/024821

§ 371 Date: Dec. 6, 2024

Related U.S. Application Data
(60) Provisional application No. 63/350,363, filed on Jun. 8, 2022.

(51) Int. Cl. G01N 33/50; A61K 31/422; A61K 45/06; A61P 35/00

(52) U.S. Cl. CPC G01N 33/5017; A61K 31/422; A61K 45/06; A61P 35/00; G01N 33/5023; G01N 33/5044; G01N 2500/10

(57) ABSTRACT

Methods of treating cancer, including methods that administer to a patient an agent that may be an inducer of cancer stem cell differentiation and mesenchymal to epithelial transition (MET) in multiple tumor cell lines in a 3D culture model that recapitulates clinically relevant tumor cell states in vitro. The differentiating activity of the agents in cancer stem cells may occur through over-activation, rather than inhibition, of the Notch pathway. Methods of screening agents, and pharmaceutical compositions.

### CROSS-REFERENCE TO RELATED APPLICATIONS

This application claims the benefit of U.S. Provisional Patent Application No. 63/350,363, filed Jun. 8, 2022, which is incorporated by reference herein in its entirety.

### STATEMENT REGARDING FEDERALLY SPONSORED RESEARCH AND DEVELOPMENT

This invention was made with government support under MCB 1651855, awarded by the National Science Foundation (NSF). The government has certain rights in the invention.

### BACKGROUND

Despite significant therapeutic advances, cancer remains a major public health issue and a critical barrier to increasing life expectancy in most countries, if not every country, of the world (see, e.g., Bray, F. et al. Cancer 127 (16): 3029-30)). In 2019, the World Health Organization (WHO) estimated that cancer is the first or second leading cause of death before the age of 70 years in most countries worldwide. In the United States, cancer is the second leading cause of mortality, and in California, more people are estimated to die of cancer than in any other state (Siegel, R. L. et al. CA: A Cancer Journal for Clinicians 71 (1): 7-33).

The development of surgery, radiotherapy, chemotherapy, and targeted therapy has reduced the cancer death rate over recent years, but these treatments are only effective in a subset of malignant tumors (Sun, Y. et al. 2015, Medicinal Research Reviews 35 (2): 408-36). Frequently, they fail to prevent or treat recurrence and metastasis, typically due to cancer heterogeneity, resistance to chemotherapy and radiotherapy, avoidance of immunological surveillance, or a combination thereof (Batlle, E. et al. 2017, Nature Medicine 23 (10): 1124-34). Cancer stem cells (CSCs) can explain most, if not all, of these failure mechanisms (Reya, T., S. et al. 2001, Nature 414 (6859): 105-11).

CSCs constitute a small subpopulation of tumor cells that are directly responsible for tumorigenesis, and can generate heterogeneous populations of tumorigenic and nontumorigenic cancer cell progeny through the processes of self-renewal and differentiation (see, e.g., Lapidot, T. et al. 1994, Nature 367 (6464): 645-48). Evidence demonstrates a strong causal role for CSCs in recurrence, metastasis, multidrug resistance, and radiation resistance of multiple tumor types (see, e.g., Kang, Mi K. et al. 2008, BMC Neuroscience 9 (January): 15). Therefore, the development of CSC-targeted therapeutic strategies may hold significant promise for improving survival and/or quality of life in patients with cancer. According to some researchers, eradication of CSCs through differentiation, arrest, and/or targeted killing may be required for successful cancer therapy (Dingli, D. et al. 2006, “Successful Therapy Must Eradicate Cancer Stem Cells.” Stem Cells).

While CSCs may be a broad cancer phenomenon, aggressive cancers that have reached advanced stages upon initial diagnosis may benefit most from CSC targeted therapy, likely because these cancers do not respond well to current therapies. For example, pancreatic ductal adenocarcinoma (PDAC) is one of the most aggressive solid tumors, and is characterized by a five-year survival rate less than 8%; its remarkable resistance to treatment is likely conferred by pancreatic CSCs (Di Carlo, C. et al. 2018, World Journal of Stem Cells 10 (11): 172-82). Triple negative breast cancer (TNBC) is also highly aggressive, lacks targeted therapy options, and is associated with a higher risk of early metastasis and poorer outcomes than other breast cancer subtypes (Park, S. et al. 2019, Cancers 11 (7)). These two diseases also disproportionately impact people in minority populations (see, e.g., Vick, Alexis D., et al. 2019, Pancreas 48 (2): 242-49).

Cancer progression, from tumor initiation to metastatic spread, is a complex process that requires cells to adjust their cell state depending on the time and environment. Due to the inherent heterogeneity of tumors, it is generally recognized that a fraction of cells within a tumor, i.e., the cancer stem cells, are endowed with the self-renewal capabilities and cancer-propagating ability to drive tumor progression. Similarly, the transition from non-metastatic to metastatic cancer is likely driven by invasion of single cells and/or cell collectives which undergo partial epithelial-to-mesenchymal transition (EMT) and maintain a hybrid E-M cell state that allows cancer cells to switch between phenotypes throughout the metastatic cascade (e.g., local detachment and invasion, intravasation, circulation, extravasation and distant metastasis).

Importantly, it has been reported that EMT can induce the formation of cancer stem cells and that these two cancer progression programs are in fact linked. Differentiation therapy is a method of chemotherapy that induces differentiation of the cancer stem cell fraction of cells within a heterogeneous tumor.

After several decades of research into CSC biology, a few CSC-targeting strategies have advanced to the preclinical stage. However, only modest therapeutic effects have been observed in several aggressive cancers including TNBC but not PDAC (Park, So-Yeon, et al. 2019, Cancers 11 (7); Subramaniam, D. et al. 2018, Current Medicinal Chemistry 25 (22): 2585-94; Saygin, C. et al. 2019, Cell Stem Cell 24 (1): 25-40).

A key challenge may be that the majority of currently available strategies inhibit developmental signaling pathways that regulate the maintenance and survival of both normal SCs and CSCs, e.g., Notch, Wnt, Hedgehog. Therefore, the therapeutic window of these approaches remains unclear. A more comprehensive understanding of CSC-specific targets and optimization of dosing may improve these strategies.

There remains a need for improved methods and agents for treating cancer, including, but not limited to, therapeutic strategies that effectively target CSCs, such as therapeutic strategies that target CSCs for MET and differentiation while strategically avoiding effects on normal SCs.

### BRIEF SUMMARY

Provided herein are improved methods and compositions for treating cancer, embodiments of which include administering agents that are potent inducers of CSC differentiation and/or mesenchymal to epithelial transition (MET) in multiple tumor cell lines, including PDAC and TNBC, in a 3D culture model that recapitulates clinically relevant tumor cell states in vitro, as described herein. It was surprisingly discovered, as explained in the Examples provided herein, that the differentiating activity of embodiments of the inducers in CSCs occurred through over-activation, rather than inhibition, of the Notch pathway, which is reminiscent of dose-dependent behaviors observed previously (Mazzone, M. et al. 2010, Proceedings of the National Academy of Sciences of the United States of America 107 (11): 5012-17). In other words, it has been surprisingly discovered that pharmacological activation of Notch signaling by agents, such as Isoxazole (ISX) derivatives, can induce and/or promote cancer cell (e.g., cancer stem cell) differentiation (e.g., terminal differentiation and/or differentiation into a progenitor cell) across multiple cancer types, including breast cancer, fibrosarcoma, liver cancer, bone cancer, bladder cancer, lung cancer, sarcoma, skin cancer, colon/colorectal cancer, lymphoma, leukemia, ovarian cancer, cervical cancer, pancreatic cancer, brain cancer and gastric cancer.

Therefore, embodiments of the agents, i.e., inducers, described herein represent a distinct therapeutic strategy. Also described herein is a mechanism of action (MOA) of the inducers in CSCs, which is distinct from normal SCs. As a result, embodiments of the inducers may effectively target CSCs for MET and differentiation while strategically avoiding effects on normal SCs.

In one aspect, methods of treatment are provided, such as methods of treating cancer. In some embodiments, the methods include administering to a patient an amount of a Notch pathway activator, a RAP1 pathway activator, a RhoA pathway activator, or a combination thereof. The amount of the Notch pathway activator, the RAP1 pathway activator, the RhoA pathway activator, or the combination thereof is effective to (i) induce mesenchymal to epithelial transition (MET) of one or more cells or cell types, such as CSCs, and/or sensitivity to chemotherapy, (ii) inhibit proliferation, maintenance, survival, and/or viability of cancer stem cells (CSC), (iii) inhibit epithelial to mesenchymal transition (EMT), proliferation, maintenance, survival, and/or viability of non-CSC tumor cells, or (iv) a combination thereof.

In another aspect, methods of screening are provided as a research tool, such as methods of screening Notch pathway activator candidates, RAP1 pathway activator candidates, and/or RhoA pathway activator candidates. In some embodiments, the methods include providing a cell culture in a three-dimensional (3D) high-density collagen matrix, a patient-derived xenograft (PDX) model, or a patient-derived organoid (PDO) model, wherein the cell culture may include cells in a Notch-activated state conferred at least in part by the 3D high-density collagen matrix, the PDX model, or the PDO model, and wherein the cells of the cell culture exhibit a first expression of one or more Notch pathway genes, RAP1 pathway genes, and/or RhoA pathway genes; contacting the cell culture and a Notch pathway activator candidate, a RAP1 pathway activator candidate, and/or a RhoA pathway activator candidate to form a treated cell culture; determining a second expression of the one or more Notch pathway genes, RAP1 pathway genes, and/or RhoA pathway genes exhibited by the cells of the treated cell culture; and determining whether the second expression is greater than the first expression, wherein the Notch pathway activator candidate, the RAP1 pathway activator candidate, and/or the RhoA pathway activator candidate is a Notch pathway activator, a RAP1 pathway activator, and/or a RhoA pathway activator, respectively, if the second expression is greater than the first expression.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 depicts Notch pathway gene expression in an embodiment of a model system provided herein and other common in vitro model systems.

FIG. 2 depicts microscopy images of certain cancer cell lines at 7 days of culture in an embodiment of a high-density collagen matrix treated with an embodiment of an agent.

FIG. 3A depicts the results of a gene expression analysis of Notch target genes.

FIG. 3B depicts immunofluorescence of cell nuclei and cleaved intracellular domain of Notch1.

FIG. 3C depicts a series of invasion distances.

FIG. 3D depicts Epithelial-to-Mesenchymal (EMT) status using western blot (WB) analysis.

FIG. 3E depicts RT-qPCR of CD24 expression of embodiments of treated cells.

FIG. 3F and FIG. 3G depicts flow cytometry analysis of embodiments of cells.

FIG. 3H depicts relative CD24 mRNA expression of embodiments of cells.

FIG. 4A depicts flow cytometry analysis of CD44/CD24 expression in MDAs treated with vehicle or ISX9 for 7 days in 2D TC.

FIG. 4B depicts flow cytometry analysis of fluorescence profiles of CD44/CD24 expression in MDAs treated with vehicle or ISX9 for 7 days in HD Col1.

FIG. 4C depicts a flow cytometry analysis of CD44⁺/CD24⁻ MDA cells treated with vehicle or ISX9.

FIG. 4D depicts a western blot analysis of EMT markers E-cadherin and Vimentin in MDAs treated with vehicle or ISX9 for 7 days in 2D TC or HD Col1.

FIG. 4E depicts a western blot analysis of EMT transcription factor TWIST1 in LL/2 lung cancer cells treated with vehicle or ISX9 for 7 days in 2D TC or HD Col1.

FIG. 4F depicts flow cytometry distributions of CDH1 expression in MDAs treated with vehicle or ISX9 for 7 days in 2D TC, LD Col1, MD Col1, or HD Col1 conditions.

FIG. 4G depicts a flow cytometry analysis of CDH1⁺ MDA cells treated with vehicle or ISX9 in 2D TC, LD Col1, MD Col1, or HD Col1.

FIG. 5 depicts flow cytometry distributions of CDH1 expression in MDAs treated with vehicle or ISX9 for 7 days in 2D TC, LD Col1, MD Col1, or HD Col1 conditions.

FIG. 6 depicts the results of the screening of isoxazole analogs for MDA differentiation.

FIG. 7A depicts the results of a test demonstrating that ISX9-treated MDAs acquire increased sensitivity to treatment with doxorubicin.

FIG. 7B depicts the results of a test demonstrating that ISX9-treated R40Ps acquire increased sensitivity to treatment with gemcitabine.

FIG. 7C depict the metabolic activity, cytotoxicity, and relative CD24 expression dose-response to ISX9.

FIG. 8A depicts a western blot assessment of canonical EMT markers, Vimentin, and E-Cadherin for two TNBC PDX tumors PIM025 and PA-14-13.

FIG. 8B depicts histological sections of TNBC PDX models displaying nuclear/cytoplasmic TWIST1 colocalization in PIM025 and primary nuclear TWIST1 localization in PA-14-13.

FIG. 8C depicts SHG imaging of Collagen I stromal architectures in TNBC PDX sections of PIM025 and PA-14-13.

FIG. 8D depicts RT-qPCR of CD24 and CDH1 expression in PIM025 PDOs treated with vehicle or ISX9.

FIG. 8E depicts RT-qPCR of CD24 and CDH1 expression in PA-14-13 PDOs treated with vehicle or ISX9.

FIG. 8F and FIG. 8G depict a flow cytometry analysis of CD44⁺/CD24⁻ CSC in PIM025 PDOs treated with vehicle or ISX9, respectively.

FIG. 8H and FIG. 8I depict a flow cytometry analysis of CD44⁺/CD24⁻ CSC in PA-14-13 PDOs treated with vehicle or ISX9, respectively.

FIG. 8J and FIG. 8K depict a flow cytometry analysis of CDH1⁺ cells in PIM025 PDOs treated with vehicle or ISX9, respectively.

FIG. 8L and FIG. 8M depict a flow cytometry analysis of CDH1⁺ cells in PA-14-13 PDOs treated with vehicle or ISX9, respectively.

FIG. 8N depicts a western blot analysis of E-Cadherin and Vimentin in PIM-025 PDOs treated with vehicle or ISX9 for 6 days.

FIG. 8O depicts a western blot analysis of E-Cadherin and Vimentin in PA-14-13 PDOs treated with vehicle or ISX9 for 6 days.

FIG. 8P and FIG. 8Q depict results of tests showing that PIM-025 PDOs and PA-14-13 PDOs, respectively, pre-treated with ISX9 acquire increased sensitivity to doxorubicin.

FIG. 9 depicts similar abundances of collagen I (COL1A1) stomal microarchitectures, low E-Cadherin (CDH1) expression, and chain-like cancer cell patterns found within the tumor of a breast cancer patient.

FIG. 10A depicts differentially expressed genes identified from PhenoSeq that are related to intracellular iron usage.

FIG. 10B depicts genes whose expression decreases when Tfrc is knocked down, and are enriched in the invasive phenotype.

FIG. 10C depicts histopathology of breast cancer patients from HPA, which revealed that patient expression profiles were consistent with invasive (#1910) and non-invasive (#4193) iron-related gene sets.

FIG. 11A and FIG. 11B depict western blot analyses of 2D- and 3D-treated MDA-MB-231 cells for 24 hours.

FIG. 11C and FIG. 11D depict western blot analysis showing an increase in acetylation in patient-derived organoid models via treatment of PIM025 and PA-14-13, respectively.

FIG. 12A and FIG. 12B depict the results of the treatment of MDA-MB-231 with ISX9 for 48 hours.

### DETAILED DESCRIPTION

Provided herein are methods of treating a patient having cancer, such as carcinoma. The patient may be any animal, such as a mammal, e.g., a human. The methods may include administering to a patient an amount of a Notch pathway activator, a RAP1 pathway activator, a RhoA pathway activator, or a combination thereof. When a combination of these agents is administered, the agents may be present at any ratio. For example, the methods may include administering to a patient a Notch pathway activator, or a Notch pathway activator and a RAP1 pathway activator, wherein any ratio of the Notch pathway activator to the RAP1 pathway activator may be administered to the patient.

The amount of a Notch pathway activator, a RAP1 pathway activator, a RhoA pathway activator, or a combination thereof administered to a patient (in one or more doses) may be effective to (i) induce mesenchymal to epithelial transition (MET) of one or more cells or cell types, such as CSCs, and/or sensitivity to chemotherapy, (ii) inhibit proliferation, maintenance, survival, and/or viability of CSCs, (iii) inhibit epithelial to mesenchymal transition (EMT), proliferation, maintenance, survival, and/or viability of non-CSC tumor cells, or (iv) a combination thereof.

In some embodiments, the methods include providing a Notch pathway activator, a RAP1 pathway activator, a RhoA pathway activator, or a combination thereof. The providing of a Notch pathway activator, a RAP1 pathway activator, a RhoA pathway activator, or a combination thereof may include providing or forming (i) a pharmaceutical composition that includes a Notch pathway activator, a RAP1 pathway activator, a RhoA pathway activator, or a combination thereof, or (ii) a drug delivery device or system that includes a Notch pathway activator, a RAP1 pathway activator, a RhoA pathway activator, or a combination thereof. As described herein, any known drug delivery device or system may be used in the administering of the one or more agents, and the pharmaceutical compositions may be configured for any effective route of administration.

In some embodiments, the providing of a Notch pathway activator, a RAP1 pathway activator, a RhoA pathway activator, or a combination thereof includes screening the one or more agents, such as by any of the screening methods described herein.

In some embodiments, the providing of the Notch pathway activator, the RAP1 pathway activator, the RhoA pathway activator, or the combination thereof may include providing a cell culture in a three-dimensional (3D) high-density collagen matrix. The providing of the cell culture may include collecting cells from a patient, and culturing the cells in a 3D high-density collagen matrix, such as any of those described herein. The providing of the cell culture may include collecting cells from a patient, and culturing the cells in mice (e.g., PDX models) or in organoids (e.g., PDO models). The cell culture may include cells in a Notch-activated state conferred at least in part by a 3D high-density collagen matrix, a PDX model, or a PDO model. The cell culture may include a plurality of cells, which may include cancer stem cells (CSCs) and non-CSC tumor cells. The plurality of cells, such as the cancer stem cells, may exhibit a first expression of one or more Notch pathway genes, RAP1 pathway genes, RhoA pathway genes or a combination thereof. The methods also may include contacting the cell culture and a Notch pathway activator candidate, a RAP1 pathway activator candidate, a RhoA pathway activator candidate, or a combination thereof to form a treated cell culture; determining a second expression of the one or more Notch pathway genes, RAP1 pathway genes, RhoA pathway genes, or a combination thereof exhibited by the plurality of cells of the treated cell culture; determining whether the second expression is greater than the first expression, wherein the Notch pathway activator candidate, the RAP1 pathway activator candidate, and/or the RhoA pathway activator candidate is the Notch pathway activator, the RAP1 pathway activator, and/or the RhoA pathway activator, respectively, if the second expression is greater than the first expression. The genes may include any of those described herein, including, but not limited to, RBPJ, JAG1, LFNG, NOTCH1, NOTCH2, HES1, or a combination thereof.

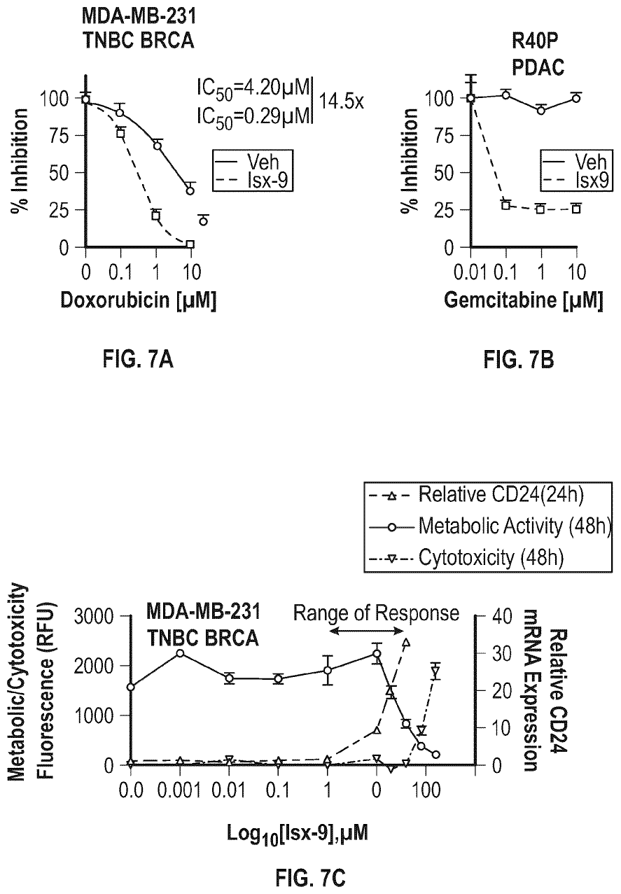

The methods described herein may include administering one or more additional therapies and/or subjecting the patient to one or more procedures (see FIG. 7). In some embodiments, the methods may also include administering to the patient a chemotherapy, radiation therapy, a hormone ablation therapy, a pro-apoptosis therapy, an immunotherapy, a cancer therapy or agent, or a combination thereof prior to, concurrently with, and/or after administration of the Notch pathway activator, the RAP1 pathway activator, and/or the RhoA pathway activator. The cancer therapy or agent may target rapidly dividing cells, disruption of cell cycle, cell division, or a combination thereof. In some embodiments, the methods include performing surgery on the patient prior to, concurrently with, and/or after administration of a Notch pathway activator, a RAP1 pathway activator, and/or a RhoA pathway activator. The surgery may include any of those used in cancer treatment, such as tumor resection. In some embodiments, the patient has a carcinoma that includes bulk cancer cells that are not cancer stem cells, and the cancer therapy or agent is capable of killing or inhibiting the bulk cancer cells. The frequency of the bulk cancer cells may be reduced.

The agents described herein (e.g., a Notch pathway activator, a RAP1 pathway activator, and/or a RhoA pathway activator) may be administered to a patient in any manner using any known technique. For example, the administering to the patient of the Notch pathway activator may include transiently administering the Notch pathway activator, such as by lipofection, a nanoparticle delivery system, or a nanogel delivery system.

The patients treated and/or screened by the methods described herein may have cancer of any type, such as carcinoma. The carcinoma may be a metastatic carcinoma. The cancer may include one or more cancer stem cells (CSCs) selected from the group consisting of a breast CSC, a fibrosarcoma CSC, a pancreatic CSC, a liver CSC, a brain CSC, a melanoma CSC, a lung CSC, a T-cell acute lymphoblastic leukemia (T-ALL) CSC, and a prostate CSC. The cancer may include one or more CSCs selected from the group consisting of CD44+/CD24−, CD133+, ALDH+, EpCAM+, CD24+, CD90+, and CD49f+.

### Compounds, Including Isoxazole Derivatives

In some embodiments, the Notch pathway activator, RAP1 pathway activator, and/or RhoA pathway activator is an isoxazole derivative. In some embodiments, the Notch pathway activator, the RAP1 pathway activator, the RhoA pathway activator, the Notch pathway activator candidate, the RAP1 pathway activator candidate, and/or the RhoA pathway activator candidate is a compound of formula (I), or a pharmaceutically acceptable salt thereof:

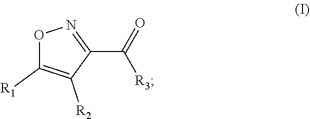

wherein R₁ is selected from the group consisting of a substituted or unsubstituted phenyl, a substituted or unsubstituted thiophenyl, and a substituent of formula (A)—

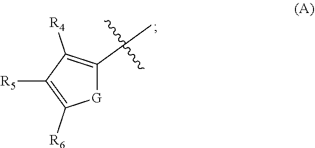

wherein R₄, R₅ and R₆, independently, are selected from the group consisting of hydrogen, hydroxy, halo, cyano, nitro, unsubstituted or substituted alkyl (C≤10), unsubstituted or substituted aryl (C≤12), unsubstituted or substituted aralkyl (C≤15), unsubstituted or substituted heteroaryl (C≤12), and unsubstituted or substituted acyl (C≤10); wherein G is O, NH, or S; wherein R2 is selected from the group consisting of hydrogen, hydroxy, halo, nitro, unsubstituted or substituted alkyl (C≤10), unsubstituted or substituted alkenyl (C≤10), unsubstituted or substituted alkynyl (C≤10), unsubstituted or substituted alkoxy (C≤10), unsubstituted or substituted alkenyloxy (C≤10), unsubstituted or substituted alkynyloxy (C≤10), unsubstituted or substituted aryl (C≤12), unsubstituted or substituted aralkyl (C≤15), unsubstituted or substituted acyl (C≤10), —C(O)R₇, —OC(O)R₇, —OC(O)OR₇, —C(O)NR₈R₉, OC(O)NR₈R₉, —NR₈OR₉, and —SO₃R₇; wherein R₇ is selected from the group consisting of hydrogen, unsubstituted or substituted alkyl (C≤10), unsubstituted or substituted aryl (C≤12), and unsubstituted or substituted aralkyl (C≤15); wherein R₈ and R₉, independently, are selected from the group consisting of hydrogen, unsubstituted or substituted alkyl (C≤10), unsubstituted or substituted aryl (C≤12), and unsubstituted or substituted aralkyl (C≤15), or together R₈ and R₉ are unsubstituted or substituted alkanediyl (C≤6); wherein R₃ is selected from the group consisting of unsubstituted or substituted —NH—O-alkyl (C≤10), —NHOH, —OR₁₀, and —NR₁₁R₁₂; wherein R₁₀ is selected from the group consisting of hydrogen, substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15); wherein R₁₁ and R₁₂, independently, are selected from the group consisting of substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15), or together R11 and R12 form alkanediyl (C≤6), —CH₂CH₂NHCH₂CH₂—, or —CH₂CH₂OCH₂CH₂—.

In some embodiments, the compound of formula (I) is a compound of formula (II):

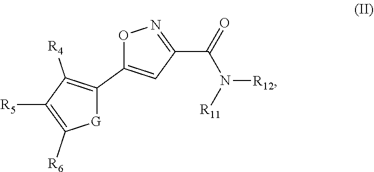

wherein R₄, R₅, R₆, R₁₁, and R₁₂ are as defined herein.

In some embodiments, R₁₁, R₁₂, or both R₁₁ and R₁₂ is/are hydrogen in formula (II). In some embodiments, R₁₁ is hydrogen, and R₁₂ is selected from the group consisting of substituted or unsubstituted alkyl (C≤10), unsubstituted alkenyl (C≤10), substituted or unsubstituted alkynyl (C≤10), and unsubstituted or substituted benzyl. In some embodiments, R₁₁ and R₁₂ together are —CH₂CH₂CH₂—, —CH₂CH₂CH₂CH₂—, —CH₂CH₂CH₂CH₂CH₂—, —CH₂CH₂NHCH₂CH₂—, or —CH₂CH₂OCH₂CH₂—. For example, when together R₁₁ and R₁₂ are —CH₂CH₂CH₂CH₂—, the compound of formula (II) includes the following moiety:

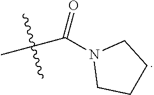 [SMILES: *C(=O)N1CCCC1]

In some embodiments, R12 of formula (II) is selected from the group consisting of a cycloalkyl, such as cyclopropyl, an aliphatic (C≤10) alcohol, and an aliphatic (C≤10) polyol.

In some embodiments, the Notch pathway activator, the RAP1 pathway activator, the RhoA pathway activator, the Notch pathway activator candidate, the RAP1 pathway activator candidate, and/or the RhoA pathway activator candidate comprises one or more of the following compounds or a pharmaceutically acceptable salt thereof:

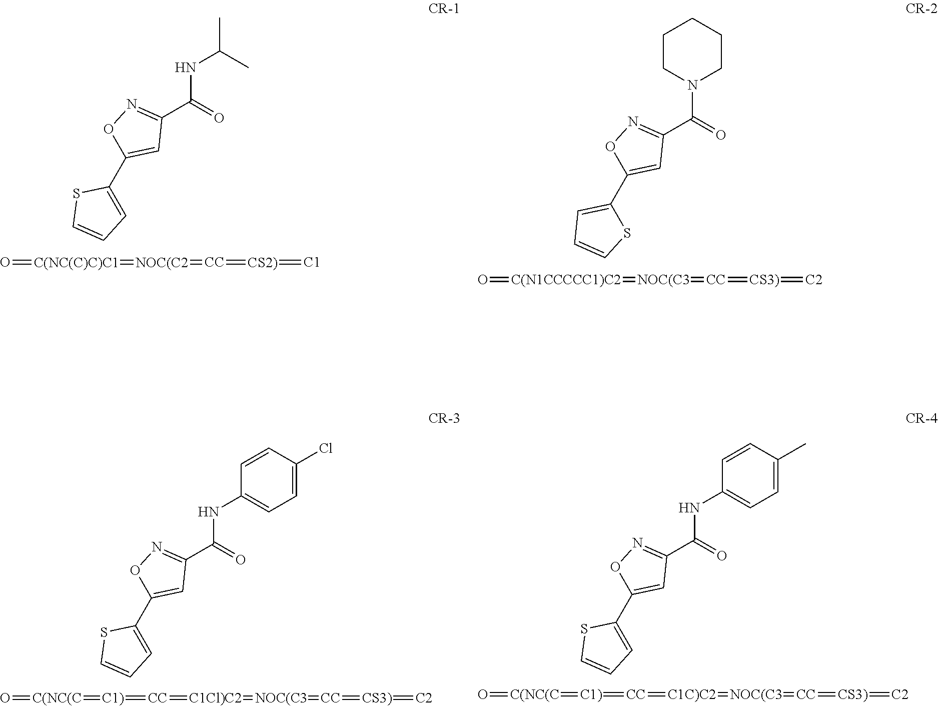

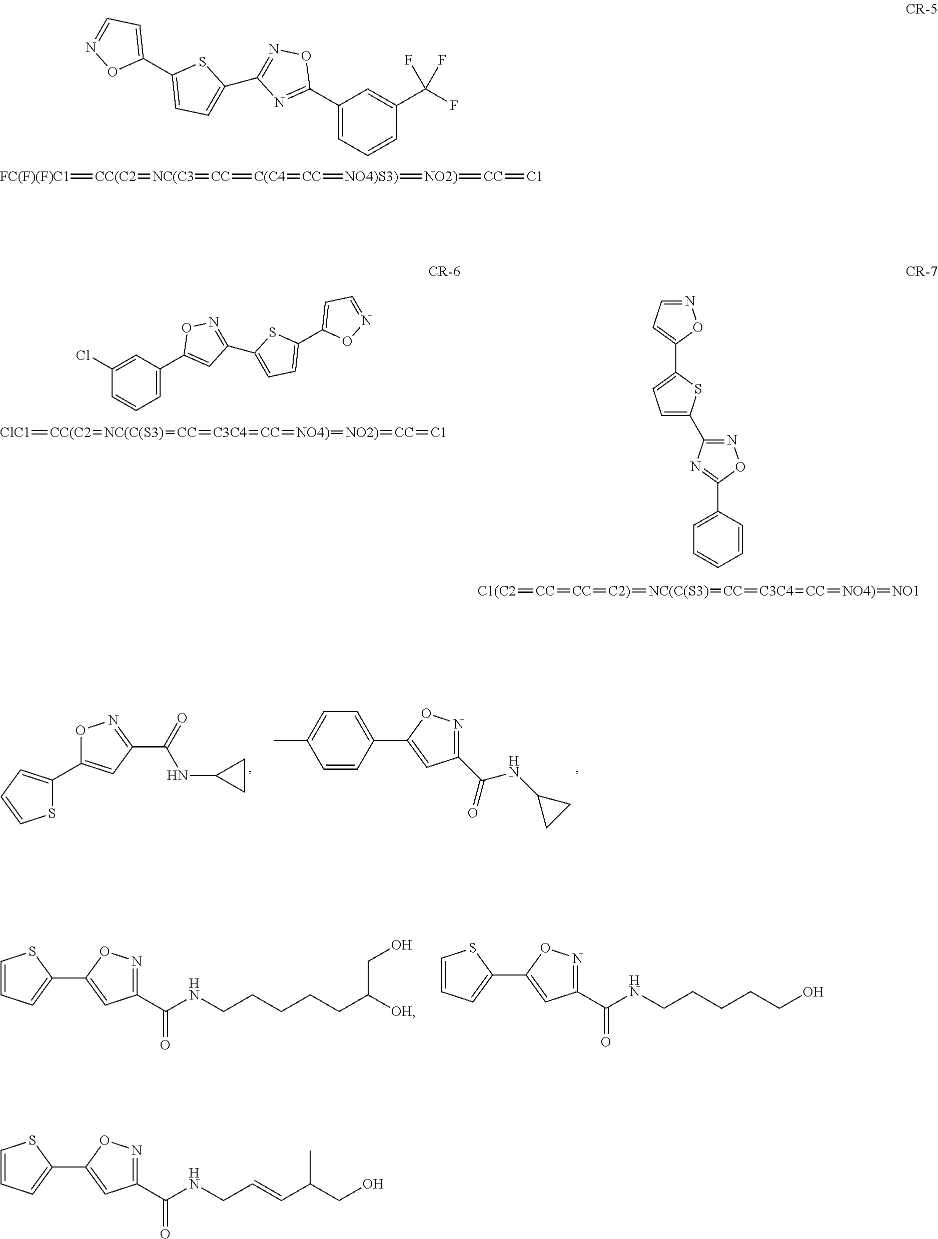

Also provided herein are pharmaceutical compositions. The pharmaceutical compositions may include a Notch pathway activator, a RAP1 pathway activator, a RhoA pathway activator, a Notch pathway activator candidate, a RAP1 pathway activator candidate, and/or a RhoA pathway activator candidate. A pharmaceutical composition may be configured for any route of delivery, such as any of those described herein. The pharmaceutical compositions may include any one or more of the components described herein, such as a carrier, which typically is a pharmaceutically acceptable carrier. The pharmaceutical compositions, in some embodiments, consist essentially of a Notch pathway activator, a RAP1 pathway activator, a RhoA pathway activator, or a combination thereof, meaning that the Notch pathway activator, the RAP1 pathway activator, and/or the RhoA pathway activator is/are the only active ingredient of the pharmaceutical composition.

### Definitions

As it would be understood, the section or subsection headings as used herein is for organizational purposes only and are not to be construed as limiting or separating or both limiting and separating the subject matter described.

Throughout this disclosure, various publications, patents and published patent specifications are referenced by an identifying citation. The disclosures of these publications, patents and published patent specifications are hereby incorporated by reference into the present disclosure in their entireties to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof. For example, the phrase “a Notch pathway activator” includes one Notch pathway activator or a combination of two or more different Notch pathway activators, for example, CR-1 and CR-2.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

Unless otherwise indicated, the term “substituted,” when used to describe a chemical structure or moiety, refers to a derivative of that structure or moiety wherein (i) a multi-valent non-carbon atom (e.g., oxygen, nitrogen, sulfur, phosphorus, etc.) is bonded to one or more carbon atoms of the chemical structure or moiety (e.g., a “substituted” C₄ alkyl may include, but is not limited to, diethyl ether moiety, a butoxy moiety, etc., and a “substituted” C₁₂ aryl may include, but is not limited to, an oxydibenzene moiety, a benzophenone moiety, etc.) or (ii) one or more of its hydrogen atoms (e.g., chlorobenzene may be characterized generally as a C₆ aryl “substituted” with a chlorine atom) is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), tertiary amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

A “composition” as used herein, refers to an active agent, such as a compound as disclosed herein and a carrier, inert or active. The carrier can be, without limitation, solid such as a bead or resin, or liquid, such as phosphate buffered saline.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo, or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

Administration or treatment in “combination” refers to administering two agents such that their pharmacological effects are manifest at the same time. Combination does not require administration at the same time or substantially the same time, although combination can include such administrations.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor, or delay, slowing, or prevent of relapse. In one aspect, treatment excludes prophylaxis. In one aspect, treatment provides a longer progression free survival or a longer overall survival. In one aspect, treatment excludes prevention.

In one embodiment, the term “disease” or “disorder” as used herein refers to a cancer or a tumor (which are used interchangeably herein), a status of being diagnosed with such disease, a status of being suspect of having such disease, or a status of at high risk of having such disease. In one aspect, the cancer is ovarian cancer such as ovarian serous carcinoma.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. In some embodiments, the term “cancer” is used interchangeably with the term “tumor”.

As used herein, an ablative therapy is a treatment destroying or ablating cancer tumors. In one embodiment, the ablative therapy does not require invasive surgery. In other embodiments, the ablative therapy refers to removal of a tumor via surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “contacting” means direct or indirect binding or interaction or physical contact between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” (DFS) indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2nd Ed., Oxford University Press (1987).

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov, last visited on May 1, 2008. Typically, a patient is given a subsequent chemotherapy regimen because the patient did not show a positive clinical or sub-clinical response to the first line therapy or the first line therapy has stopped.

An isoxazole is an azole with an oxygen atom next to the nitrogen. Isoxazoles also form the basis for a number of drugs, including the COX-2 inhibitor valdecoxib (Bextra) and a neurotransmitter agonist AMPA. A derivative, furoxan, is a nitric oxide donor. An isoxazolyl group is found in many beta-lactamase-resistant antibiotics, such as cloxacillin, dicloxacillin and flucloxacillin. Leflunomide is an isoxazole-derivative drug. Examples of AAS containing the isoxazole ring include danazol and androisoxazole. Thus, these compounds are commercially available. ISX-9 (Isoxazole 9) is a potent inducer of adult neural stem cell differentiation. The compound is commercially available from MedChemExpress (MCE), for example. The compound and its derivatives as disclosed herein can be made synthesized using the protocol found in Scheider et al. (2008) Small-molecule activation of neuronal cell fate, Nature Chemical Biology 4:408-410, and in particular the supplemental figures of this publication.

### EMBODIMENTS

The following is a listing of non-limiting embodiments of the disclosure.

Embodiment 1A. A method of treating a patient having cancer, such as carcinoma, the method comprising administering to the patient an amount of a Notch pathway activator, a RAP1 pathway activator, a RhoA pathway activator, or a combination thereof effective to (i) induce mesenchymal to epithelial transition (MET) of one or more cells or cell types, such as CSCs, and/or sensitivity to chemotherapy, (ii) inhibit proliferation, maintenance, survival, and/or viability of CSCs, (iii) inhibit epithelial to mesenchymal transition (EMT), proliferation, maintenance, survival, and/or viability of non-CSC tumor cells, (iv) induces a significant increase in acetylation of histones H3 and H4, or (v) a combination thereof.

Embodiment 1B. A method of treating a patient having cancer, such as carcinoma, the method comprising administering to the patient an amount of a compound of formula (I) or a pharmaceutically acceptable salt thereof effective to (i) induce mesenchymal to epithelial transition (MET) of one or more cells or cell types, such as CSCs, and/or sensitivity to chemotherapy, (ii) inhibit proliferation, maintenance, survival, and/or viability of CSCs, (iii) inhibit epithelial to mesenchymal transition (EMT), proliferation, maintenance, survival, and/or viability of non-CSC tumor cells, (iv) induces a significant increase in acetylation of histones H3 and H4, or (v) a combination thereof:

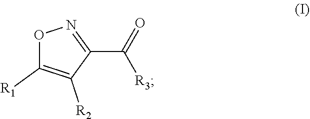

wherein R₁ is selected from the group consisting of a substituted or unsubstituted phenyl, a substituted or unsubstituted thiophenyl, and a substitutent of formula (A):

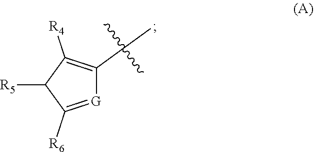

wherein R₄, R₅ and R₆, independently, are selected from the group consisting of hydrogen, hydroxy, halo, cyano, nitro, unsubstituted or substituted alkyl (C≤10), unsubstituted or substituted aryl (C≤12), unsubstituted or substituted aralkyl (C≤15), unsubstituted or substituted heteroaryl (C≤12), and unsubstituted or substituted acyl (C≤10);
wherein G is O, NH, or S;
wherein R₂ is selected from the group consisting of hydrogen, hydroxy, halo, nitro, unsubstituted or substituted alkyl (C≤10), unsubstituted or substituted alkenyl (C≤10), unsubstituted or substituted alkynyl (C≤10), unsubstituted or substituted alkoxy (C≤10), unsubstituted or substituted alkenyloxy (C≤10), unsubstituted or substituted alkynyloxy (C≤10), unsubstituted or substituted aryl (C≤12), unsubstituted or substituted aralkyl (C≤15), unsubstituted or substituted acyl (C≤10), —C(O)R₇, —OC(O)R₇, —OC(O)OR₇, —C(O)NR₈R₉, OC(O)NR₈R₉, —NR₈OR₉, and —SO₃R₇;
wherein R₇ is selected from the group consisting of hydrogen, unsubstituted or substituted alkyl (C≤10), unsubstituted or substituted aryl (C≤12), and unsubstituted or substituted aralkyl (C≤15);
wherein R₈ and R₉, independently, are selected from the group consisting of hydrogen, unsubstituted or substituted alkyl (C≤10), unsubstituted or substituted aryl (C≤12), and unsubstituted or substituted aralkyl (C≤15), or together R₈ and R₉ are unsubstituted or substituted alkanediyl (C≤6);
wherein R₃ is selected from the group consisting of unsubstituted or substituted —NH—O-alkyl (C≤10), —NHOH, —OR₁₀, and —NR₁₁R₁₂;
wherein R₁₀ is selected from the group consisting of hydrogen, substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15); and
wherein R₁₁ and R₁₂, independently, are selected from the group consisting of substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15), or together R₁₁ and R₁₂ form alkanediyl (C≤6), —CH₂CH₂NHCH₂CH₂—, or —CH₂CH₂OCH₂CH₂—.

Embodiment 2. A method of treating a patient having cancer, such as carcinoma, the method comprising (A) providing a Notch pathway activator, a RAP1 pathway activator, a RhoA pathway activator, or a combination thereof, and (B) administering to the patient an amount of the Notch pathway activator, the RAP1 pathway activator, the RhoA pathway activator, or the combination thereof effective to (i) induce mesenchymal to epithelial transition (MET) of one or more cells or cell types, such as CSCs, and/or sensitivity to chemotherapy, (ii) inhibit proliferation, maintenance, survival, and/or viability of CSCs, (iii) inhibit epithelial to mesenchymal transition (EMT), proliferation, maintenance, survival, and/or viability of non-CSC tumor cells, (iv) induces a significant increase in acetylation of histones H3 and H4, or (v) a combination thereof.

Embodiment 3. The method of Embodiment 2, wherein the providing of the Notch pathway activator, the RAP1 pathway activator, the RhoA pathway activator, or the combination thereof comprises (a) providing a cell culture (optionally grown from cells collected from the patient) in a three-dimensional (3D) high-density collagen matrix, a patient-derived xenograft (PDX) model, or a patient-derived organoid (PDO) model, wherein the cell culture may comprise cells in a Notch-activated state conferred at least in part by the 3D high-density collagen matrix, the PDX model, or the PDO model, wherein the cell culture comprises a plurality of cells (which may include cancer stem cells (CSCs) and non-CSC tumor cells), and wherein the plurality of cells, such as the cancer stem cells, exhibit a first expression of one or more Notch pathway genes, RAP1 pathway genes, RhoA pathway genes, or a combination thereof; (b) contacting the cell culture and a Notch pathway activator candidate, a RAP1 pathway activator candidate, a RhoA pathway activator candidate, or a combination thereof to form a treated cell culture; (c) determining a second expression of the one or more Notch pathway genes, RAP1 pathway genes, RhoA pathway genes, or a combination thereof exhibited by the plurality of cells of the treated cell culture; (d) determining whether the second expression is greater than the first expression, wherein the Notch pathway activator candidate, the RAP1 pathway activator candidate, and/or the RhoA pathway activator candidate is the Notch pathway activator, the RAP1 pathway activator, and/or the RhoA pathway activator, respectively, if the second expression is greater than the first expression, and (e) optionally proceeding to step (B) of Embodiment 2 if the second expression is greater than the first expression; wherein the genes may include any of those described herein, including, but not limited to, RBPJ, JAG1, LFNG, NOTCH1, NOTCH2, HES1, or a combination thereof.

Embodiment 4. A method of screening, the method comprising providing a cell culture comprising a plurality of cells in a three-dimensional (3D) high-density collagen matrix, a patient-derived xenograph (PDX) model, or a patient-derived organoid (PDO) model, wherein the cell culture may include cells in a Notch-activated state conferred at least in part by the 3D high-density collagen matrix, the PDX model, or the PDO model, and wherein the plurality of cells exhibits a first expression of one or more Notch pathway genes, RAP1 pathway genes, RhoA pathway genes, or a combination thereof; contacting the cell culture and a Notch pathway activator candidate, a RAP1 pathway activator candidate, a RhoA pathway activator candidate, or a combination thereof to form a treated cell culture; determining a second expression of one or more Notch pathway genes, RAP1 pathway genes, RhoA pathway genes, or a combination thereof exhibited by the plurality of cells of the treated cell culture; and determining whether the second expression is greater than the first expression, wherein the Notch pathway activator candidate, the RAP1 pathway activator candidate, and/or the RhoA pathway activator candidate is a Notch pathway activator, a RAP1 pathway activator, and/or a RhoA pathway activator, respectively, if the second expression is greater than the first expression; wherein the genes may include any of those described herein, including, but not limited to, RBPJ, JAG1, LFNG, NOTCH1, NOTCH2, HES1, or a combination thereof.

Embodiment 5. The method of any of the preceding embodiments, wherein the method further comprises administering a chemotherapy to the patient, wherein the chemotherapy is administered prior to, concurrently with, and/or after the administering of the Notch pathway activator, the RAP1 pathway activator, and/or the RhoA pathway activator.

Embodiment 6. The method of any of the preceding embodiments, wherein the method further comprises administering a radiation therapy to the patient, wherein the radiation therapy is administered prior to, concurrently with, and/or after administration of the Notch pathway activator, the RAP1 pathway activator, and/or the RhoA pathway activator.

Embodiment 7. The method of any of the preceding embodiments, wherein the method further comprises performing surgery on the patient prior to, concurrently with, and/or after administration of the Notch pathway activator, the RAP1 pathway activator, and/or the RhoA pathway activator.

Embodiment 8. The method of Embodiment 7, wherein the surgery comprises tumor resection.

Embodiment 9. The method of any of the preceding embodiments, wherein the method further comprises administering a hormone ablation therapy to the patient, wherein the hormone therapy is administered prior to, concurrently with, and/or after the administering of the Notch pathway activator, the RAP1 pathway activator, and/or the RhoA pathway activator.

Embodiment 10. The method of any of the preceding embodiments, wherein the method further comprises administering a pro-apoptosis therapy to the patient, wherein the pro-apoptosis is administered prior to, concurrently with, and/or after administration of the Notch pathway activator, the RAP1 pathway activator, and/or the RhoA pathway activator.

Embodiment 11. The method of any of the preceding embodiments, wherein the method further comprises administering an immunotherapy to the patient, wherein the immunotherapy is administered prior to, concurrently with, and/or after administration of the Notch pathway activator, the RAP1 pathway activator, and/or the RhoA pathway activator.

Embodiment 12. The method of any of the preceding embodiments, wherein the method further comprises administering a cancer therapy or agent to the patient, wherein the cancer therapy or agent is administered prior to, concurrently with, and/or after administration of the Notch pathway activator, the RAP1 pathway activator, and/or the RhoA pathway activator, wherein, optionally, the cancer therapy or agent targets rapidly dividing cells, disruption of cell cycle, cell division, or a combination thereof.

Embodiment 13. The method of any of the preceding embodiments, wherein the carcinoma comprises bulk cancer cells that are not cancer stem cells, and, optionally, wherein the method further comprises administering a cancer therapy or agent to the patient prior to, concurrently with, and/or after administration of the Notch pathway activator, the RAP1 pathway activator, and/or the RhoA pathway activator, wherein the cancer therapy or agent is capable of killing or inhibiting the bulk cancer cells, and wherein the frequency of the bulk cancer cells may be reduced.

Embodiment 14. The method of any of the preceding embodiments, wherein the administering to the patient of the Notch pathway activator, the RAP1 pathway activator, and/or the RhoA pathway activator comprises transiently administering the Notch pathway activator, the RAP1 pathway activator, and/or the RhoA pathway activator, such as by lipofection, a nanoparticle delivery system, or a nanogel delivery system.

Embodiment 15. The method of any of the preceding embodiments, wherein the carcinoma is a metastatic carcinoma.

Embodiment 16. The method of any of the preceding embodiments, wherein the cancer comprises one or more cancer stem cells (CSCs) selected from the group consisting of a breast CSC, a fibrosarcoma CSC, a pancreatic CSC, a liver CSC, a brain CSC, a melanoma CSC, a lung CSC, T-ALL CSC, and a prostate CSC.

Embodiment 17. The method of any of the preceding embodiments, wherein the cancer comprises one or more CSCs selected from the group consisting of CD44+/CD24−, CD133+, ALDH+, EpCAM+, CD44+, CD24+, CD90+, and CD49f+.

Embodiment 18. The method of any of the preceding embodiments, wherein the Notch pathway activator, the RAP1 pathway activator, the RhoA pathway activator, the Notch pathway activator candidate, the RAP1 pathway activator candidate, and/or the RhoA pathway activator candidate is a compound of formula (I), or a pharmaceutically acceptable salt thereof:

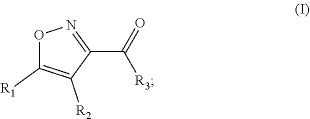

wherein R₁ is selected from the group consisting of a substituted or unsubstituted phenyl, a substituted or unsubstituted thiophenyl, and a substitutent of formula (A):

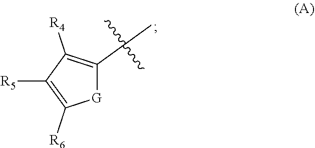

wherein R₄, R₅ and R₆, independently, are selected from the group consisting of hydrogen, hydroxy, halo, cyano, nitro, unsubstituted or substituted alkyl (C≤10), unsubstituted or substituted aryl (C≤12), unsubstituted or substituted aralkyl (C≤15), unsubstituted or substituted heteroaryl (C≤12), and unsubstituted or substituted acyl (C≤10);
wherein G is O, NH, or S;
wherein R₂ is selected from the group consisting of hydrogen, hydroxy, halo, nitro, unsubstituted or substituted alkyl (C≤10), unsubstituted or substituted alkenyl (C≤10), unsubstituted or substituted alkynyl (C≤10), unsubstituted or substituted alkoxy (C≤10), unsubstituted or substituted alkenyloxy (C≤10), unsubstituted or substituted alkynyloxy (C≤10), unsubstituted or substituted aryl (C≤12), unsubstituted or substituted aralkyl (C≤15), unsubstituted or substituted acyl (C≤10), —C(O)R₇, —OC(O)R₇, —OC(O)OR₇, —C(O)NR₈R₉, OC(O)NR₈R₉, —NR₈OR₉, and —SO₃R₇;
wherein R₇ is selected from the group consisting of hydrogen, unsubstituted or substituted alkyl (C≤10), unsubstituted or substituted aryl (C≤12), and unsubstituted or substituted aralkyl (C≤15);
wherein R₈ and R₉, independently, are selected from the group consisting of hydrogen, unsubstituted or substituted alkyl (C≤10), unsubstituted or substituted aryl (C≤12), and unsubstituted or substituted aralkyl (C≤15), or together R₈ and R₉ are unsubstituted or substituted alkanediyl (C≤6);
wherein R₃ is selected from the group consisting of unsubstituted or substituted —NH—O-alkyl (C≤10), —NHOH, —OR₁₀, and —NR₁₁R₁₂;
wherein R₁₀ is selected from the group consisting of hydrogen, substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15); and
wherein R₁₁ and R₁₂, independently, are selected from the group consisting of substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15), or together R₁₁ and R₁₂ form alkanediyl (C≤6), —CH₂CH₂NHCH₂CH₂—, or —CH₂CH₂OCH₂CH₂—.

Embodiment 19. The method of any of the preceding embodiments, wherein G is S.

Embodiment 20. The method of any of the preceding embodiments, wherein any one of R₄, R₅, or R₆ is hydrogen, any two of R₄, R₅, or R₆ are hydrogen, or each of R₄, R₅, and R₆ is hydrogen.

Embodiment 21. The method of any of the preceding embodiments, wherein R₂ is hydrogen.

Embodiment 22. The method of any of the preceding embodiments, wherein R₃ is —NR₁₁R₁₂.

Embodiment 23. The method of Embodiment 22, wherein R₁₁ or R₁₂, independently, are selected from the group consisting of cyclopropyl, cyclobutyl, cyclopentyl, and cyclohexyl.

Embodiment 24. The method of Embodiment 22, wherein together R₁₁ and R₁₂ are cycloalkyl, such as cyclopropyl, cyclobutyl, cyclopentyl, or cyclohexyl.

Embodiment 25. The method of any of the preceding embodiments, wherein the compound of formula (I) is a compound of formula (II):

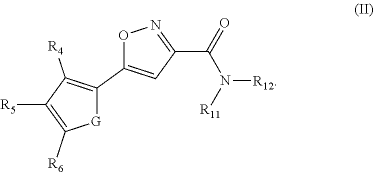

Embodiment 26. The method of Embodiment 25, wherein R₁₁, R₁₂, or both R₁₁ and R₁₂ is/are hydrogen.

Embodiment 27. The method of Embodiment 25, wherein R₁₁ is hydrogen, and R₁₂ is selected from the group consisting of substituted or unsubstituted alkyl (C≤10), unsubstituted alkenyl (C≤10), substituted or unsubstituted alkynyl (C≤10), and unsubstituted or substituted benzyl.

Embodiment 28. The method of Embodiment 25, wherein together R₁₁ and R₁₂ are —CH₂CH₂CH₂—, —CH₂CH₂CH₂CH₂—, —CH₂CH₂CH₂CH₂CH₂—, —CH₂CH₂NHCH₂CH₂—, or —CH₂CH₂OCH₂CH₂—.

Embodiment 29. The method of any of the preceding embodiments, wherein R₁₂ is selected from the group consisting of a cycloalkyl, such as cyclopropyl, an aliphatic (C≤10) alcohol, and an aliphatic (C≤10) polyol.

Embodiment 30. The method of any of the preceding embodiments, wherein the Notch pathway activator, the RAP1 pathway activator, the RhoA pathway activator, the Notch pathway activator candidate, the RAP1 pathway activator candidate, and/or the RhoA pathway activator candidate comprises one or more of the following compounds or a pharmaceutically acceptable salt thereof:

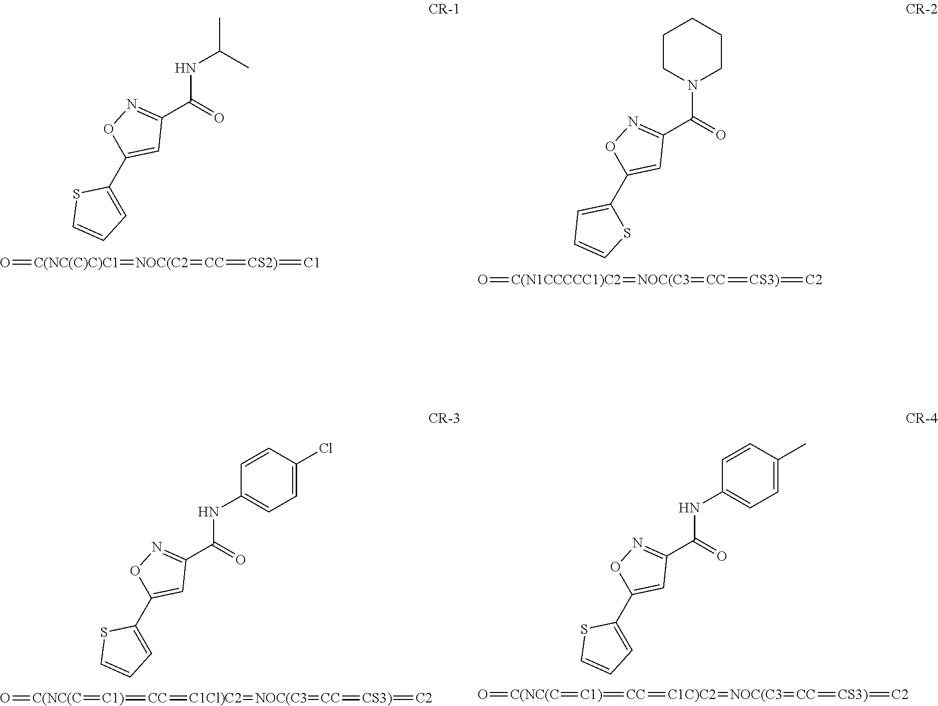

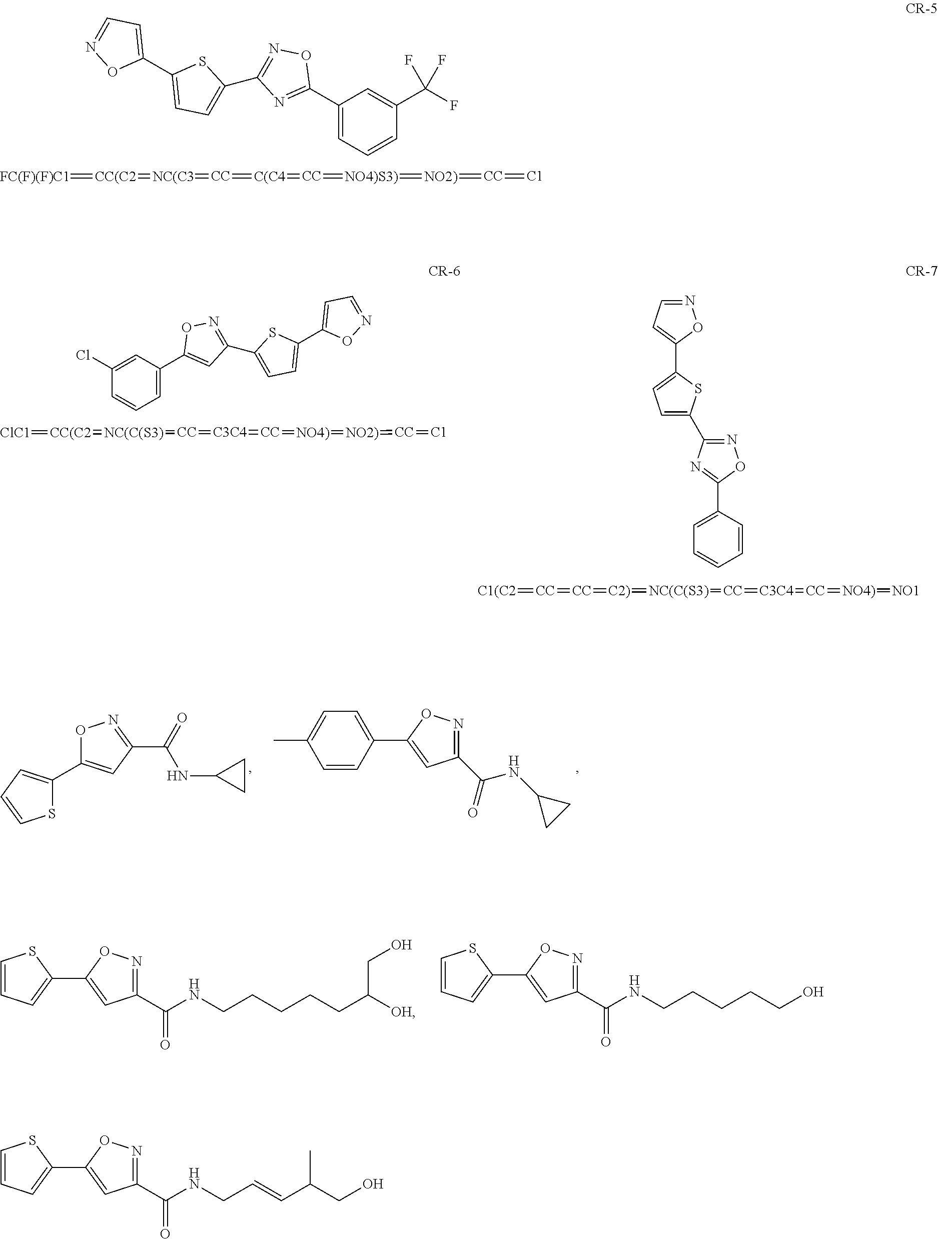

Embodiment 31. A pharmaceutical composition comprising or consisting essentially of the Notch pathway activator, the RAP1 pathway activator, the RhoA pathway activator, the Notch pathway activator candidate, the RAP1 pathway activator candidate, and/or the RhoA pathway activator candidate of any of the preceding embodiments, wherein, optionally, the pharmaceutical composition is configured for transient delivery.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

The following examples describe the surprising discovery that the agents, such as Isoxazole derivatives, can induce and/or direct activation of Notch signaling, which was believed to be a novel mechanism of action for this subset of compounds. While previous studies in solid tumors such as breast cancer, brain cancer, ovarian cancer, non-small-cell lung cancer (NSCLC), colorectal cancer, melanoma, pancreatic cancer, cholangiosarcoma and medulloblastoma have suggested an oncogenic and/or a stem-cell renewal role for Notch signaling, the following examples and the rest of this disclosure described methods to activate Notch signaling in solid tumors such that it can actually promote differentiation of cancer cells. Not wishing to be bound by any particular theory, it is believed that a novel mechanism of action (MOA) of ISX derivatives is described that occurs independently of previously studied MOAs of ISX, including activation of the pH-sensing receptor OGR1, activation of the phospholipase C (PLC)/Ca2+/PKC pathway, and/or activation of N-Methyl-D-aspartate (NMDA) receptor.

As explained in the following examples and not wishing to be bound by any particular theory, ISX appeared to induce activation of the Notch receptor via cleavage and release of the intracellular domain (NICD), which translocated to the nucleus and resulted in downstream events, including transcriptional and epigenetic changes, that ultimately induced differentiation of cancer cells. Again, not wishing to be bound by any particular theory, there, additionally or alternatively, may be one or more other possible mechanisms by which ISX9 is activating Notch1 signaling. For example, Nortch1 can interact with beta-catenin and GSK3B, which may affect its nuclear localization, stability, and/or transcriptional activities. ISX9 may be perturbing these interactions to drive nuclear Notch1 activity.

In certain cell types, differentiation of cancer stem cells was identified by observing a decrease in cells positive for the cancer stem cell markers CD44+/CD24−, the reversal of the malignant process of epithelial-mesenchymal transition (EMT), and a decrease in overall cell proliferation. Additionally, it was found that treatment with ISX of multiple cell types in native 3D Collagen I environments halted collective cell invasion.

### Example 1—Development of Model System

This example describes the development of an embodiment of a model system that replicates clinically relevant human cancer cell states associated with poor prognosis. There has been a long felt, but unmet need for preclinical models that can recapitulate tumor heterogeneity in terms of the cellular states associated with human disease so that the measured effect of candidate therapeutics is more clinically relevant and likely to translate (see, e.g., Saygin, C. et al. Cell Stem Cell 24, 25-40 (2019)).

It was determined whether culture of fibrosarcoma (HT1080) and TNBC (MDA-MB-231) cells in 3D high-density collagen 1 matrices (HD Col1) could induce cell states more closely matching clinical tumors, since this system mimics more features of advanced solid tumor microenvironments than traditional in vitro model systems.

Both cell lines were highly aggressive in terms of tumorigenicity and metastasis in mice. Further, MDA-MB-231s (MDAs) were considered highly enriched for CSCs, with over 90% of the total population expressing CD44⁺/CD24^{low/-2}. Sequencing revealed that both cancer cell types upregulated a common set of 70 genes in high-density collagen 1 (HD Col1), and collectively migrated and proliferated to form invasive networks, which were referred to as a collagen induced network phenotype (CINP).

A small percentage of cells formed non-invasive spheroids, revealing phenotypic heterogeneity that was not observed in other in vitro model systems. Importantly, the collagen-induced 70 gene module was predictive of poor prognosis in breast cancer (Table 1) and eight additional cancer types (Table 2), with evidence at the protein level demonstrated through histology (see, e.g., Velez, D. O. et al. Nat. Commun. 8, 1651 (2017)).

TABLE 1
CINP score potential to predict prognosis in stage I patients
from metabric database broken down by molecular subtype

| Metabric | | | | |
|---|---|---|---|---|
| molecular | | Death | | |
| subtype | Patient count | observed | HR | Cox p |
| Luminal B | 126 | 33 | 1.2461 | 0.3194 |
| Luminal A | 202 | 34 | 1.5996 | 0.0162 |
| Triple negative | 63 | 14 | 3.8537 | 0.0070 |
| HER2+ | 39 | 13 | 0.7152 | 0.3405 |

Analysis of CINP score potential to predict prognosis in stage I patients from METABRIC database broken down by molecular subtype

TABLE 2
TCGA pan cancer analysis independent of stage

| | | Death | | |
|---|---|---|---|---|
| Cancer type | Patient count | observed | HR | Cox p |
| LGG | 508 | 92 | 1.8434 | 1.1E−13 |
| ACC | 79 | 25 | 3.1863 | 2.8E−04 |
| CESC | 304 | 60 | 1.6560 | 5.2E−04 |
| MESO | 85 | 28 | 1.6101 | 6.9E−04 |
| PAAD | 178 | 59 | 1.5948 | 2.2E−03 |
| BLCA | 409 | 111 | 1.3338 | 0.0053 |
| LUAD | 521 | 124 | 1.2448 | 0.0169 |
| KICH | 64 | 8 | 2.9277 | 0.0210 |

Table sowing results from Kaplan-Meier and hazard ratio analysis across all cancer types in TCGA, where the CINP gene score was a significant predictor of prognosis (p < 0.05)

The results of this example indicated that HD Col1 induced multiple solid tumor cell types to adopt states that were representative of aggressive clinical disease (see, e.g., U.S. Patent Application Publication No. 20190293630 A1).

Gene ontology analysis of the CINP cell state revealed enrichment of genes involved in several biological processes, including blood vessel development and regulation of migration (see, e.g., Velez, D. O. et al. Nat. Commun. 8, 1651 (2017)). Within these two process categories, and relevant to cancer stemness, were several Notch pathway members.

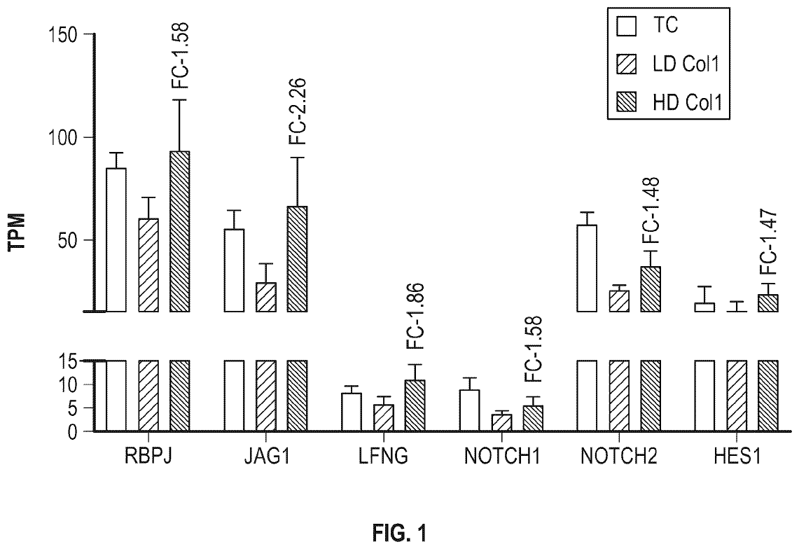

Expression of RBPJ, JAG1, LFNG, NOTCH1, NOTCH2, and HES1 in HD Col1 was distinct compared to other typical culture systems (FIG. 1). Together, this data indicated that HD Col1 induced a distinctive Notch state (FIG. 1) that was associated with more aggressive clinical disease (Tables 1-2). FIG. 1 depicts Notch pathway gene expression in the HD Col1 model system of this example versus other common in vitro model systems; specifically, a comparison of Notch pathway gene expression in MDAs cultured on tissue culture plastic (TC), in low density collagen 1 (LD Col1) (2.5 mg/mL), or in high-density collagen 1 (HD Col1) (6 mg/mL). Fold-Change (FC) was calculated in the HD Col1 with respect to the LD Col1 condition.

### Example 2—Targeting Clinically Relevant Cancer Cell States for Differentiation and MET

It has been shown that multipotent notch-activated adult stem cells respond to isoxazole derivatives by differentiating into a more precursor-like state, and these compounds are currently being explored for regenerative medicine purposes (see, e.g., Russell, J. L., et al. ACS Chem. Biol. 7, 1067-1076 (2012).

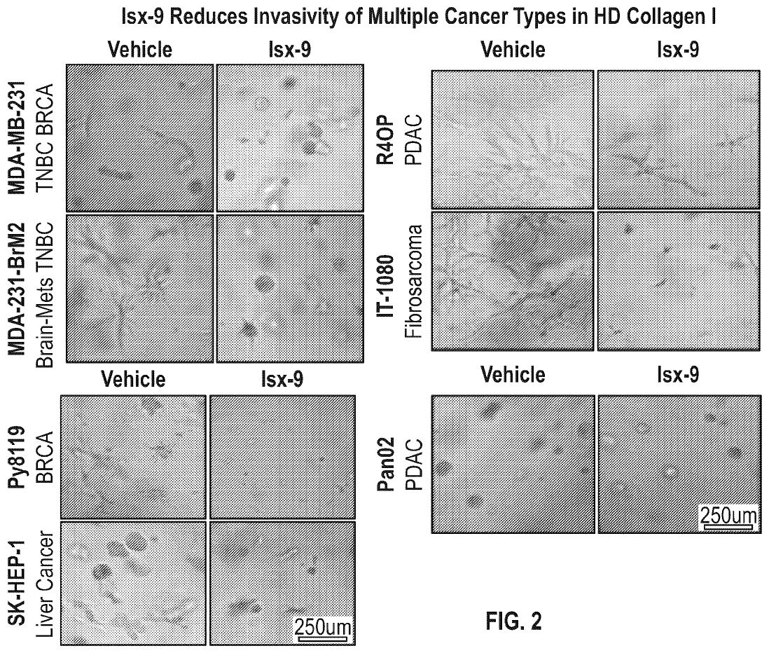

In this example, it was explored whether isoxazole derivatives could similarly induce differentiation of CSCs occupying the clinically relevant notch-activated state conferred by the HD Col1 model system of Example 1 (Table 1, FIG. 1). Treatment of a panel of aggressive cancer cell lines that formed invasive networks in the 3D model system with isoxazole derivative ISX9 showed significant abrogation of the invasive capability of MDAs and MDA-231-BrM2 (TNBC), Py8119 (Breast Cancer), HT-1080 (fibrosarcoma), Sk-HEP-1 (liver cancer), and R40P (PDAC) cells in our model system (FIG. 2). FIG. 2 depicts the results of a phenotype screen for anti-invasive activity of ISX9 in the HD Col1 model system of Example 1. FIG. 2 includes brightfield microscopy images of cancer cell lines at 7 days of culture in HD Col1 treated with vehicle or ISX9 (scale bar=250 μm).

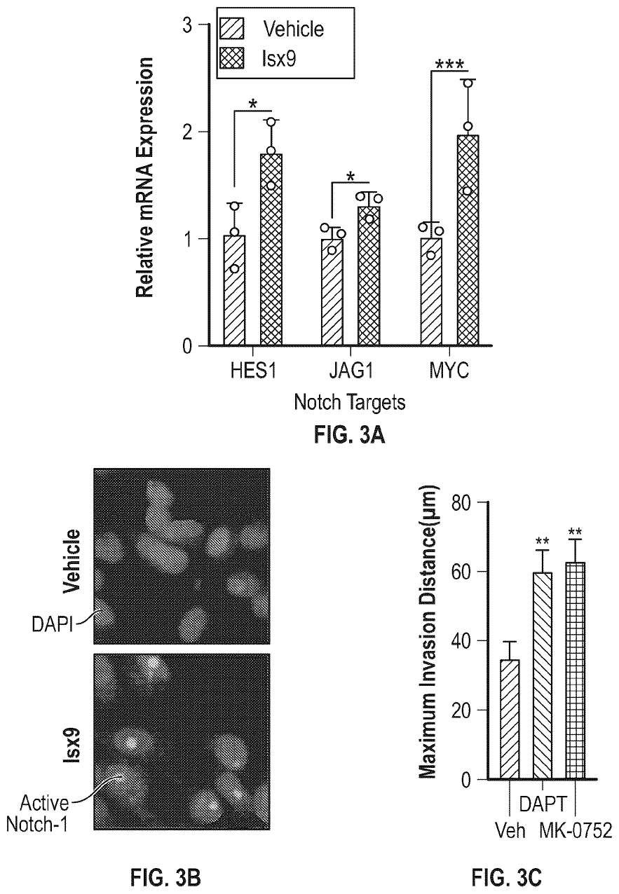

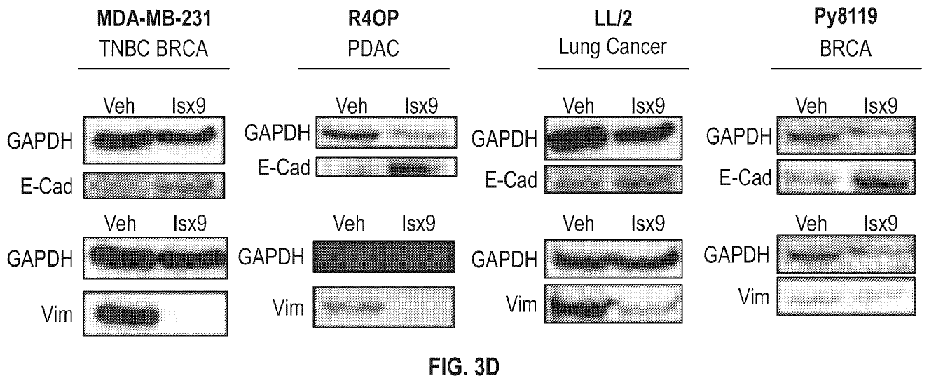

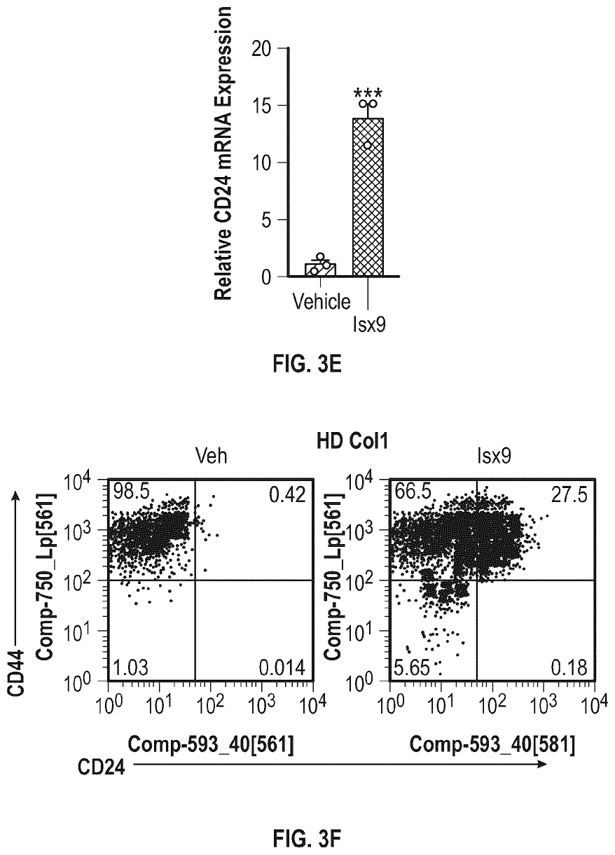

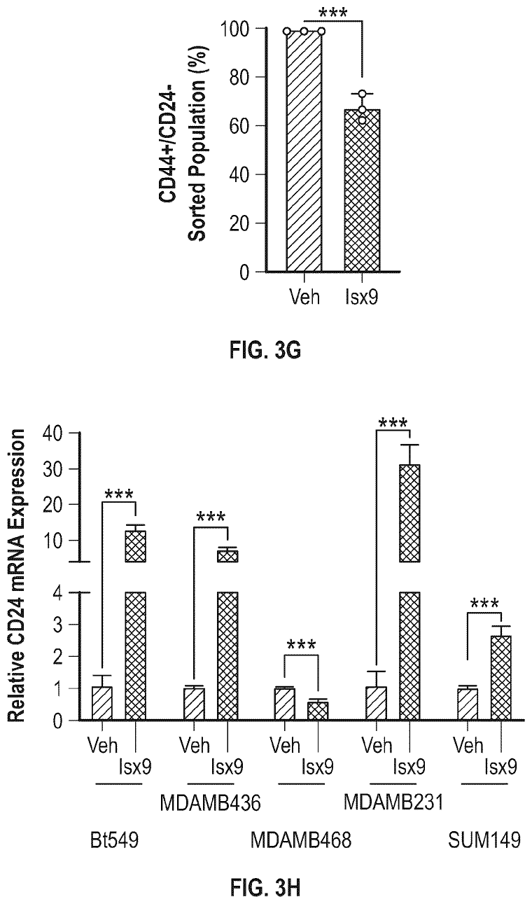

Also assessed was how the Notch pathway responded to ISX9 treatment. MD As treated with ISX9 for 24h in HD Col1 significantly increased their expression of Notch pathway genes (FIG. 3A). FIG. 3A depicts the results of a RT-qPCR gene expression analysis of Notch target genes in vehicle and Isx9 treated MDAs.

Based on the Notch expression profiles of MDAs in the model system of Example 1 (FIG. 1), it was hypothesized that Notch1 could be involved in this response.

Immunofluorescent staining for activated Notch1, i.e., the cleaved intracellular domain, showed strong nucleolar localization in ISX9 treated cells compared to vehicle treated cells, even in 2D culture (FIG. 3B). FIG. 3B depicts immunofluorescence staining of cell nuclei and cleaved intracellular domain of Notch1 in vehicle and Isx9 treated MDAs.

This result was surprising and unexpected, because current Notch-based CSC therapies focus on inhibiting the pathway, e.g., gamma-secretase inhibitors (GSIs). However, the clinical efficacy of these inhibitors has been modest to null so far (see, e.g., Yang, L. et al. Signal Transduct Target Ther. 5, 8 (2020)).

Therefore, this example assessed what effect GSIs would have on MDAs in HD Col1. DAPT and MK0752 did not inhibit the CINP, but rather slightly promoted tumor cell invasion (FIG. 3C). FIG. 3C depicts invasion distances of MDAs in HD Col1 treated with vehicle (0.1% DMSO) or GSIs DAPT and MK0752.

Given the strong phenotypic changes induced by ISX9 in HD Col1 (FIG. 2), this example also assessed the differentiation activity of ISX9 by characterizing Epithelial-to-Mesenchymal (EMT) status using western blot (WB) analysis of E-cadherin and vimentin levels. FIG. 3D shows that ISX9 treatment increased E-cadherin expression and reduced Vimentin expression after 7 days in HD Col1, suggesting that ISX9 induced MET and differentiation. FIG. 3D depicts WB of EMT markers E-cadherin and Vimentin in cancer cell lines treated with vehicle or ISX9.

To further assess differentiation status, RT-PCR and flow cytometry (FC) was used to quantify expression of the CSC stemness biomarker signature CD44⁺/CD24⁻ in MDAs. At the population level, MDAs increased CD24 mRNA expression ˜14-fold with ISX9 treatment after 7 days (FIG. 3E). FIG. 3E depicts RT-qPCR of CD24 expression in MD As treated with vehicle or ISX9. FC showed that CD44⁺/CD24⁻ cells dropped from ˜100% of the population to ˜67% of the population with ISX9 treatment. (FIG. 3F, FIG. 3G). FIG. 3F and FIG. 3G depicts flow cytometry analysis of MDAs treated with vehicle or ISX9.

Together, these data suggested that ISX9 was a promising therapeutic candidate for induction of MET and differentiation in CSCs. Other isoxazole derivatives and Notch1 agonists could function similarly, so a high-throughput screening of isoxazole derivatives was conducted, it also was determined whether these MET and differentiation readouts occurred in 2D culture at 48 h.

As expected, CD24 mRNA was significantly upregulated at 48h (FIG. 3H), which suggested that this readout could serve as a marker of early response in high-throughput screening experiments. FIG. 3H depicts relative CD24 mRNA expression. CD24 expression was an early indicator of response to ISX9 in 2D across several TNBC cell lines. Treatment screening of several different TNBC cell lines with ISX9 in 2D for 48h yielded four responders, as determined by CD24 mRNA expression (FIG. 3H). The BT-549 (FC˜12.6), MDA-MB-436 (FC˜7.1), MDAs (FC˜31.6) and SUM149 (FC˜2.7) all showed significant upregulation of CD24, but the MDA-MB-468 (FC˜0.6) showed a significant but small downregulation of CD24.

Interestingly, the two cell lines with the least response to ISX9, SUM149, and MDA-MB-468, contained Notch1 mutations that would have been expected to change the functionality of the protein (see, e.g., DepMap: The cancer dependency map project at Broad Institute). Together, this data suggested that the ability of ISX9 to differentiate CSCs may rely on its ability to over activate Notch1 signaling and is broadly applicable across TNBC cell lines but is not universal. Further characterized was the MOA of ISX9, and predicted were responders v. non responders in translational studies, which could help direct patient selection in future clinical trials.

### Example 3—Testing of Isoxazole Derivatives

Investigated in this example was whether tumor cells in 2D v. HD Collagen I responded differently to isoxazole treatment by assessing the populational expression of CD44⁺/CD24⁻ CSC cells.

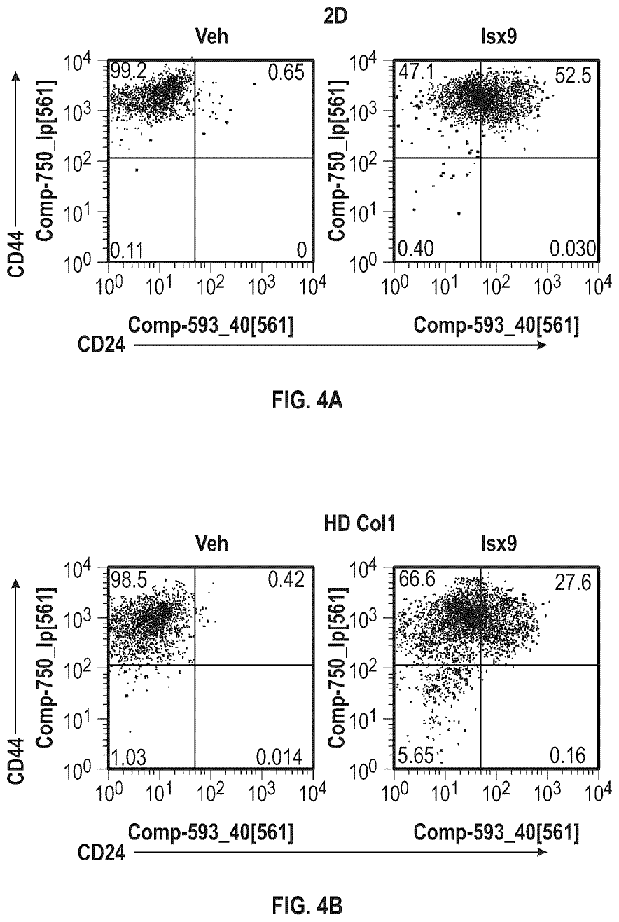

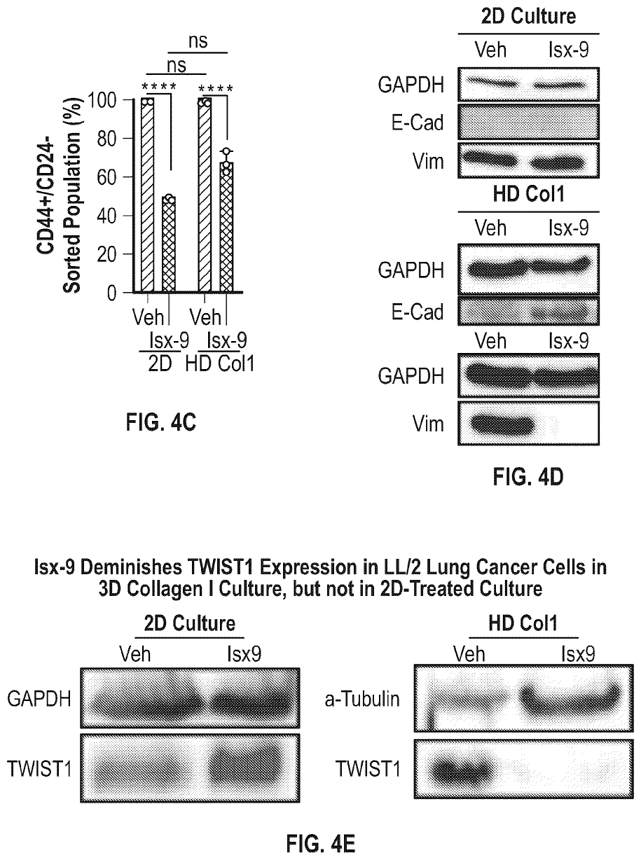

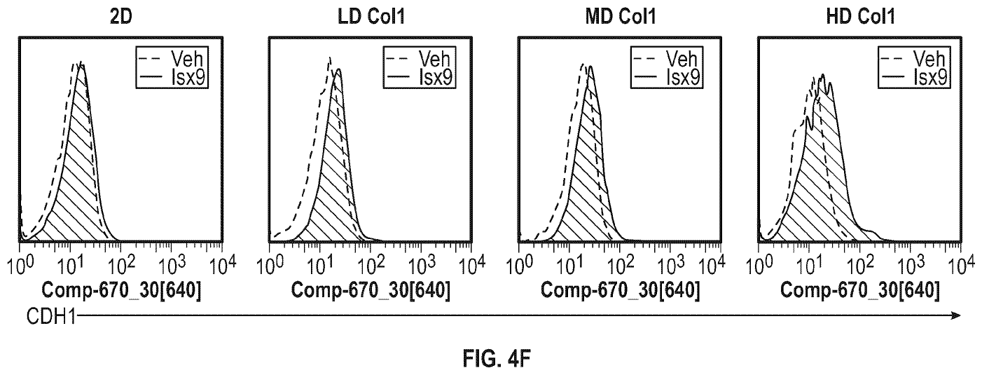

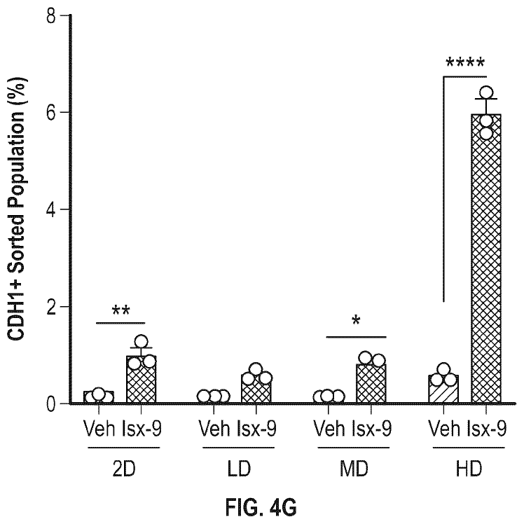

Cells in 2D tissue culture and HD Collagen I were treated with ISX9 daily for 7 days, dissociated and flow-sorted. Fluorescence-activated cell sorting (FACS) analysis showed that cells treated in 2D and HD Collagen I were able to upregulate CD24+ expression, thereby reducing the population of CD44⁺/CD24⁻ CSCs to a similar degree (FIG. 4A, FIG. 4B, and FIG. 4C). These figures depict fluorescence profiles of CD44/CD24 in MDAs treated with vehicle or ISX9 for 7 days in (FIG. 4A) 2D TC or (FIG. 4B) HD Col1. FIG. 4C depicts a flow cytometry analysis of CD44⁺/CD24⁻ MDA cells treated with vehicle or ISX9.

To understand whether ISX9 treatment could induce Mesenchymal-to-Epithelial Transition (MET), western blotting (WB) was performed for the epithelial marker E-Cadherin (CDH1) and the Mesenchymal marker Vimentin (Vim) on 2D and HD Collagen-treated tumor cells (FIG. 4D). FIG. 4D depicts a western blot analysis of EMT markers E-cadherin and Vimentin in MDAs treated with vehicle or ISX9 for 7 days in 2D TC or HD Col1.

ISX9 induced an increase in E-Cadherin and decrease in Vimentin in HD Col1-treated cells. Interestingly, there was no effect on either MET marker in 2D-treated cells. When the EMT transcription factor TWIST1 was assessed, it was found that ISX9 treatment reduced TWIST1 protein expression in HD Col1 and not in 2D culture, which suggested the regulation of TWIST1 in inducing MET and differentiation (FIG. 4E). FIG. 4E depicts a western blot analysis of EMT transcription factor TWIST1 in LL/2 lung cancer cells treated with vehicle or ISX9 for 7 days in 2D TC or HD Col1.

To determine whether induction of MET was specific to the HD Collagen I condition or generally applicable to all ISX9-treated cells in a 3D Collagen condition, CDH1⁺ cells in 2D, and multiple 3D Collagen I densities were flow-sorted (1 mg/mL, 2.5 mg/mL, and 6 mg/mL Col1) (FIG. 4F, FIG. 4G).) FIG. 4F depicts fluorescence profiles of CDH1 in MDAs treated with vehicle or ISX9 for 7 days in 2D TC, LD Col1, MD Col1, or HD Col1 conditions. FIG. 4G depicts a flow cytometry analysis of CDH1⁺ MDA cells treated with vehicle or ISX9 in 2D TC, LD Col1, MD Col1, or HD Col1.

In good accordance with the WB data, cells in the HD (6 mg/mL) Col1 condition had the highest population of CDH1⁺ cells when treated with ISX9, while ISX9 treatment of cells on 2D had a small effect to CDH1⁺ cells. Also assessed were ISX9-treated cells in 1 mg/mL and 2.5 mg/mL Col1 conditions for CDH1⁺ induction, and found cells were unable to re-express CDH1⁺to similar levels as the 6 mg/mL condition. These data demonstrated that the HD Collagen I condition rendered cells into a unique transcriptional state that allowed for increased sensitivity to differentiation treatment with Isx-9.

### Example 4—MOA of ISX9 in CSCs

This example demonstrated that MOA of ISX9 in CSCs was distinct from MOA in NSCs. ISX9 was originally identified in a chemical screen for drivers of neuronal differentiation (see, e.g., Schneider, J. W. et al. Nat. Chem. Biol. 4, 408-410 (2008)). Given that nerve differentiation bears close resemblance to gut endocrine and pancreas specification, ISX9 has also been tested for differentiating activity in progenitor cells from these tissues with promising results (see, e.g., Dioum, E. M. et al. Proc. Natl. Acad. Sci. U.S.A. 108, 20713-20718 (2011); Tsakmaki, A. et al. Mol. Metab. 34, 157-173 (2020)).

In neural stem cells, ISX9 was thought to trigger Ca²⁺ signaling through voltage-gated L-type Ca²⁺ channels and NMDA receptors, which activates CaMKII, the major HDAC kinase. ISX9-induced phosphorylation of HDAC5 leads to export of HDAC5 from the neural stem cell nucleus, thereby de-repressing MEF2 and other transcription factors to directly activate MEF2 target genes such as NR₁ and indirectly activate neuroD and other neuronal genes, which together promote early phenotypic differentiation (Schneider, J. W. et al. Nat. Chem. Biol. 4, 408-410 (2008)).

ISX9 has also been shown to target cardiac progenitor cells. In these cells, a G_{q} protein-coupled receptor (G_{q}PCR) MOA hypothesis was tested in a cell-based functional screen where one agonist hit was identified, the extracellular proton/pH-sensing GPCR GPR68, and confirmed through genetic gain- and loss-of-function studies. ISX9 activation of GPR68 functions through Ca2⁺ signaling (see, e.g., Russell, J. L. et al. ACS Chem. Biol. 7, 1077-1083 (2012)).

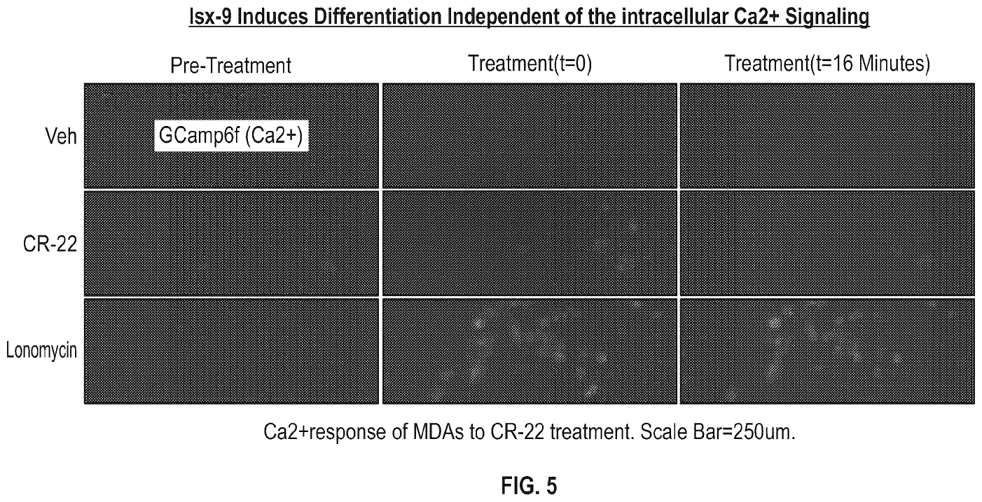

Therefore, to begin to characterize the MOA of ISX9 in CSCs, it was first determined whether ISX9 treatment of CSCs triggered Ca²⁺ signaling. MDAs were transduced to express the genetically encoded Ca²⁺ sensor GCamp6f and treated with vehicle, ionomycin (positive control), or ISX9 (data not shown). Ca²⁺ signal was observed for the positive control condition, ionomycin treatment, but not ISX9 treatment. Inhibition of phospholipase C (PLCi), one of the most common downstream effectors of Ca²⁺ signals (see, e.g., Putney, J. W. et al. Adv. Biol. Regul. 52, 152-164 (2012)), also did not counteract the phenotypic effects of ISX9 (FIG. 5). FIG. 5 depicts Ca²⁺ response of MDAs to ISX9 treatment. Scale bar=250 μm.

Finally, CSCs were treated with NMDA, the NMDA receptor agonist, and observed no phenotypic effects compared to ISX9 (data not shown). Together, these data suggested a distinct MOA in CSCs compared to NSCs.

### Example 5—SAR Expansion Screening

In this example, SAR expansion screening identified structurally similar active and inactive analogs. In order to validate that the scaffold of the phenotypic screening hit, ISX9, was an attractive candidate for chemoproteomics-enabled target identification and for further medicinal chemistry HtL optimization, a Structure Activity Relationship (SAR) expansion study was conducted by evaluating seven commercially available lead-like isoxazole analogs (labeled CR1-7) with high (>93%) substructure similarity to ISX9. These compounds had the following structures:

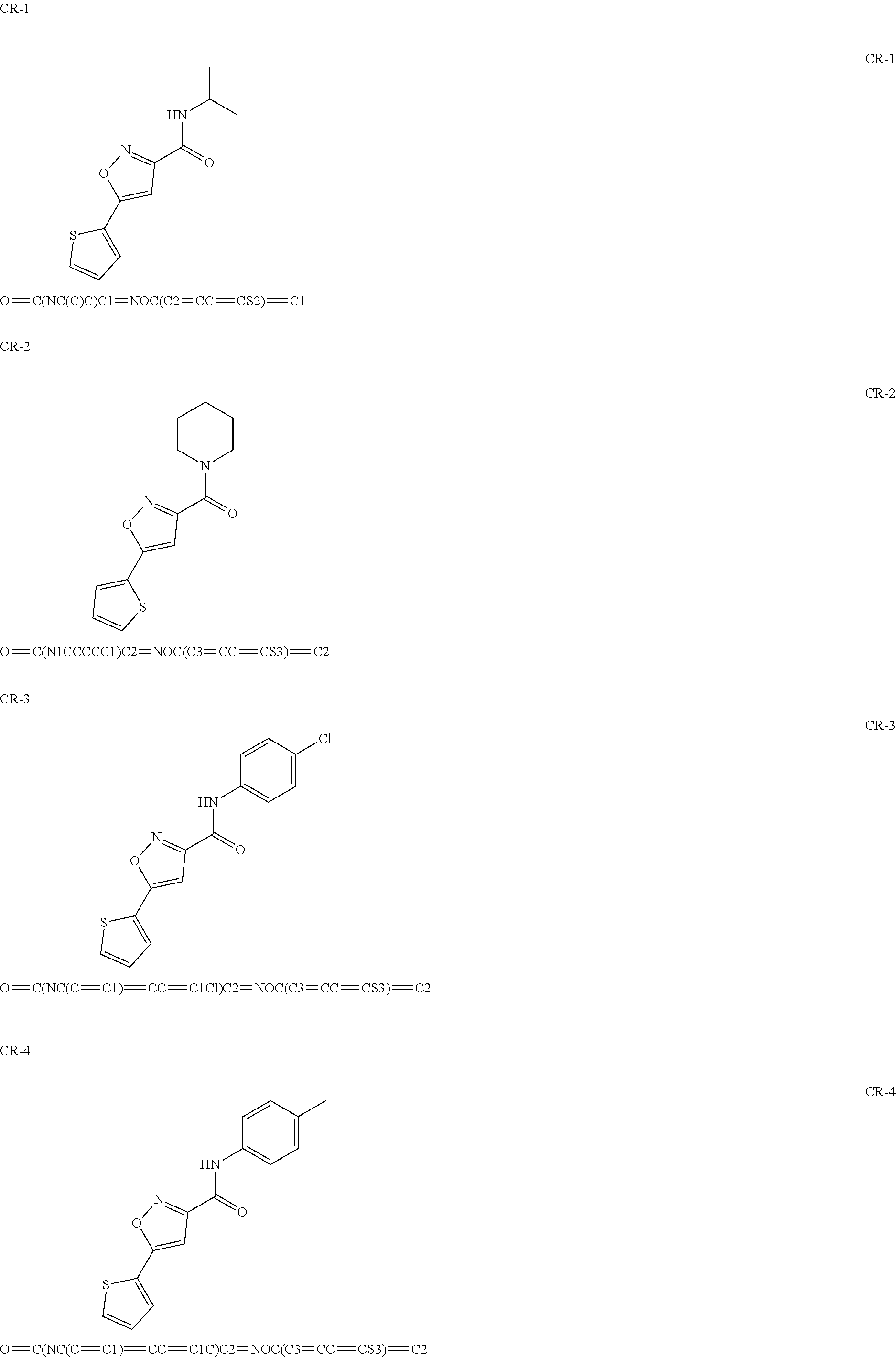

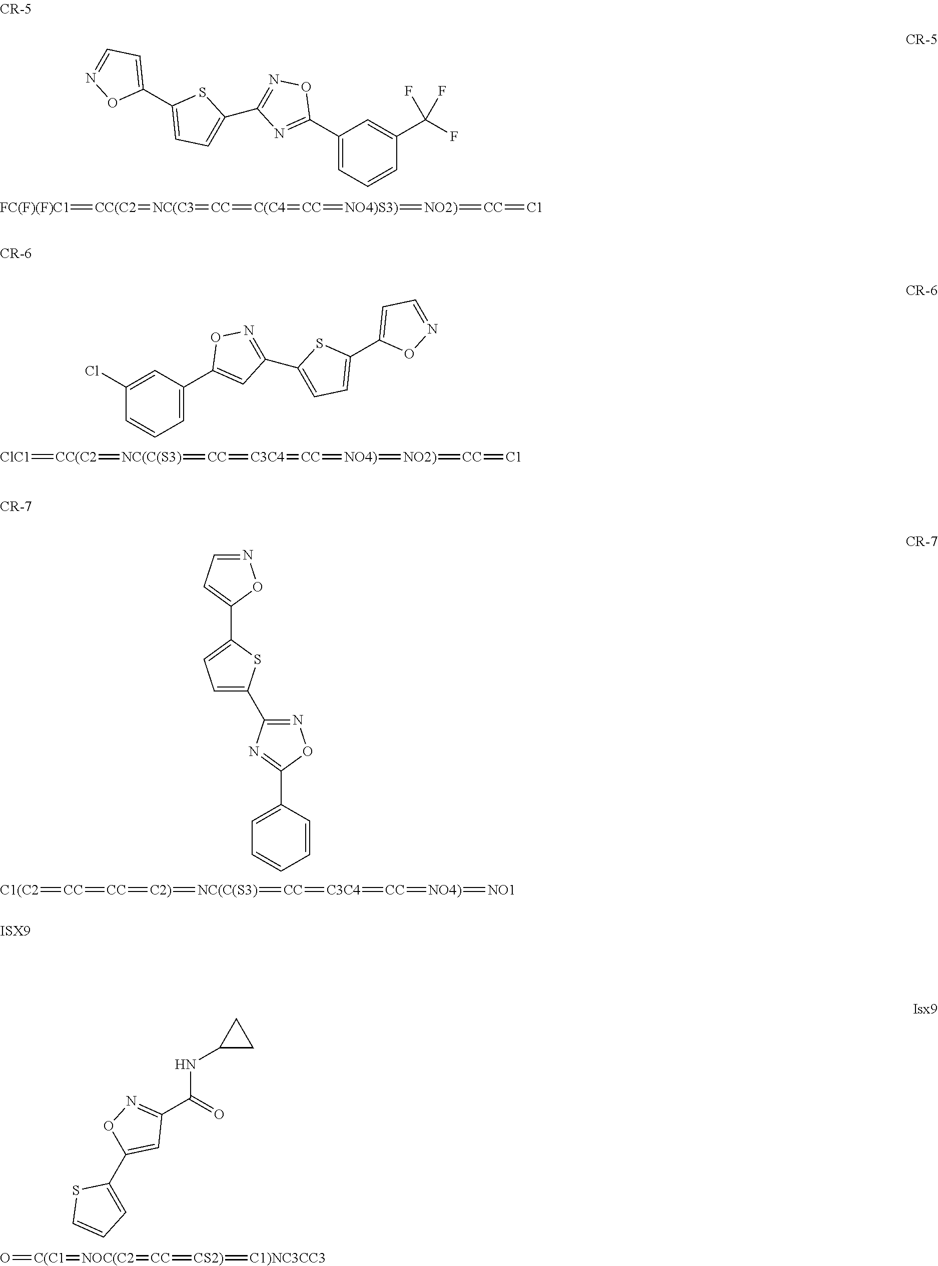

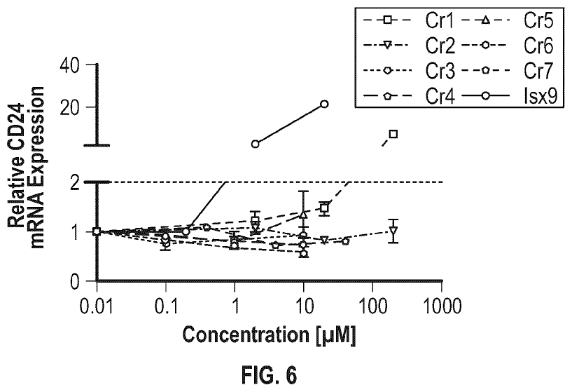

These analogs were screened for their potential to differentiate MDA-MB-231s, yielding at least one new hit, CR-1 (˜98% similarity), which induced a ˜6-fold upregulation of CD24 at a concentration of 200 μM at 48h in 2D (FIG. 6). FIG. 6 depicts the results of the screening of isoxazole analogs for MDA differentiation as measured by upregulation of CD24 mRNA (FC_{threshold}=2.0).

Six compounds with lesser or equivalent similarity to ISX9 (93-98%) did not induce CD24 expression at 48 h in 2D. This narrow range of activity within 8 highly similar analogs suggested a discontinuous, non-flat SAR with a specific target(s). As most of the sub-structural modifications from the analog screen were to the same amide R-group, this large difference in potency between analogs represented an activity cliff (AC), which can be highly beneficial when optimizing the potency of compounds described herein (see, e.g., Stumpfe, D., et al. ACS Omega 4, 14360-14368 (2019)).

Additionally, this SAR information can be used for chemoproteomics-enabled target identification to inform points of attachment for incorporating affinity tags without compromising drug activity. The observations from the SAR expansion study were distinct from the original screening conducted in NSCs where several analogs of ISX9 with high substructural similarity displayed a flat SAR for expression changes to the NSC differentiation marker neuroD, which also suggested a distinct target(s) in CSCs (see, e.g., Schneider, J. W. et al. Nat. Chem. Biol. 4, 408-410 (2008)). These results supported the hit series described herein as an excellent candidate for advancement in medicinal chemistry optimization.

### Example 6—Toleration of ISX9 In Vivo

ISX9 is well-tolerated in vivo. Prior studies assessing the activity of ISX9 in multipotent Notch-activated epicardium-derived cells (NECs) for heart regeneration after myocardial infarction (MI) lended insight into the in vivo activity of this small molecule (see, e.g., Russell, J. L. et al. ACS Chem. Biol. 7, 1067-1076 (2012)).

In adult transgenic Notch reporter mice, (TNR; CBF1-REₓ₄-EGFP) (see Russell, J. L. et al. Circ. Res. 108, 51-59 (2011)) ISX9 administered as a once daily 16 mg/kg intra-peritoneal (ip) injection for several weeks had no signs or symptoms of lethargy, weight loss or decreased appetite, and their serum biochemistry and hematology profiles were normal (see, e.g., Russell, J. L. et al. ACS Chem. Biol. 7, 1067-1076 (2012)). In pharmacokinetic surveys, ISX9 was detected in the tissue of interest to the study, the heart, even though the compound's terminal half-life in plasma after single bolus ip injection was <15 minutes (see Id.). Some of these findings were validated with a second structurally distinct isoxazole analog (see Id.). ISX9 directed muscle transcriptional programs in vivo in NECs, generating Notch-activated adult cardiomyocyte-like precursors. However, MI preemptively differentiated NECs towards fibroblast lineages, overriding ISX9's cardiogenic influence in this cell population. ISX9 dysregulated gene expression in vivo in Notch-activated repair-fibroblasts, driving distinctive pro-angiogenesis gene programs, but failed to mitigate fibrosis or avert ventricular functional decline after MI. The leaders of this study attributed failure to microenvironmental fibrosis cues overwhelming differentiation signals, which highlighted the need for in vitro model systems that put cells into physiologically relevant cell states.

Such model systems, like the HD Col1 system for solid tumor cells described herein, should help in the effort to translate in vitro-defined small-molecule bioactivities into a therapeutically beneficial in vivo functionality. Importantly, these studies indicated that ISX9 does not appear to have in vivo toxicity issues. In addition, several drugs with an isoxazole nucleus are clinically available for diverse therapeutic activities (see, e.g., Agrawal, N. et al. Med. Chem. Res. 27, 1309-1344 (2018)).

These data support the promise of using ISX9 as a Notch-activated CSC targeted therapy.

### Example 7-CSC Differentiation Therapy and Chemo-Susceptibility

CSC differentiation therapy promoted chemo-susceptibility. Differentiation therapy rendered CSCs more sensitive to a range of chemotherapeutics (see, e.g., Pattabiraman, D. R. et al. Science 351, aad3680 (2016)), and has been successfully implemented in acute promyelocytic leukemia with the combination of retinoic acid and arsenic (see, e.g., de Thé, H. Nat. Rev. Cancer 18, 117-127 (2018)).

To determine whether ISX9 could be used in a similar therapeutic function, MDAs and R40P cells were treated with vehicle or ISX9 for 7d in HD Col1, followed by chemotherapy for 3d and MTS assay to quantify viable cells. MDAs exhibited a ˜14.5-fold decrease in IC50 to doxorubicin and the R40Ps showed similar increases in chemo-sensitivity to gemcitabine, when normalized to the vehicle control (FIG. 7A, FIG. 7B). FIG. 7A demonstrated that ISX9-treated MDAs acquired increased sensitivity to treatment with doxorubicin, and FIG. 7B demonstrated that ISX9-treated R40Ps acquire increased sensitivity to treatment with gemcitabine.

To validate that the response to ISX9 did not result from off-target cytotoxicity, CellTiter-Fluor™ and CytoTox-Fluor™ assays (Promega, USA), which measure metabolic activity and cytotoxicity, respectively, were multiplexed (FIG. 7C). FIG. 7C depicts the metabolic activity, cytotoxicity, and relative CD24 expression dose-response to ISX9.

In MDAs treated with ISX9 in 2D, there was an upregulation of CD24 mRNA without any observable increase in cytotoxicity for a concentration range between 1-40 μM. Between concentrations of 10-40 μM, there was also a decrease in metabolic activity without an equivalent increase in cytotoxicity, which was consistent with cell cycle arrest from cell differentiation. These results supported the drug concentrations selected for the PDO studies.

### Example 8—Patient-Derived Models

In this example, patient-derived models recapitulated ISX9 activity. PDO and PDX models maintained molecular and architectural features of their originating patient tumor sample (see, e.g., Crystal, A. S. et al. Science 346, 1480-1486 (2014); Marangoni, E. et al. Curr. Opin. Oncol. 26, 556-561 (2014); DeRose, Y. S. et al. Nat. Med. 17, 1514-1520 (2011); Eirew, P. et al. Nature 518, 422-426 (2015); and Bruna, A. et al. Cell 167, 260-274.e22 (2016)) and predict clinical trial drug response (see, e.g., Gao, H. et al. Nat. Med. 21, 1318-1325 (2015)).

Fragmentation of patient-derived tumors into smaller organoids (PDOs) for short-term 7-day drug testing could also retain the in vivo extent of tumor cell heterogeneity and act as a representative model of a patient's response to treatment (see, e.g., Bruna, A. et al. Cell 167, 260-274.e22 (2016)). A number of TNBC PDX models in NSG mice (IACUC #S06112) can be used (Powell, R. T. et al. Sci. Rep. 10, 17899 (2020)).

Two orthotopic PDX models of TNBC were selected, the PIM-025 and PA-14-13 that represent the range of different hybrid E/M states found in TNBC (see, e.g., Grasset, E. M. et al. Sci. Transl. Med. 14, eabn7571 (2022)), as characterized by expression profiles for the conserved epithelial marker, E-Cadherin (Ecad), and mesenchymal marker, Vimentin (Vim).

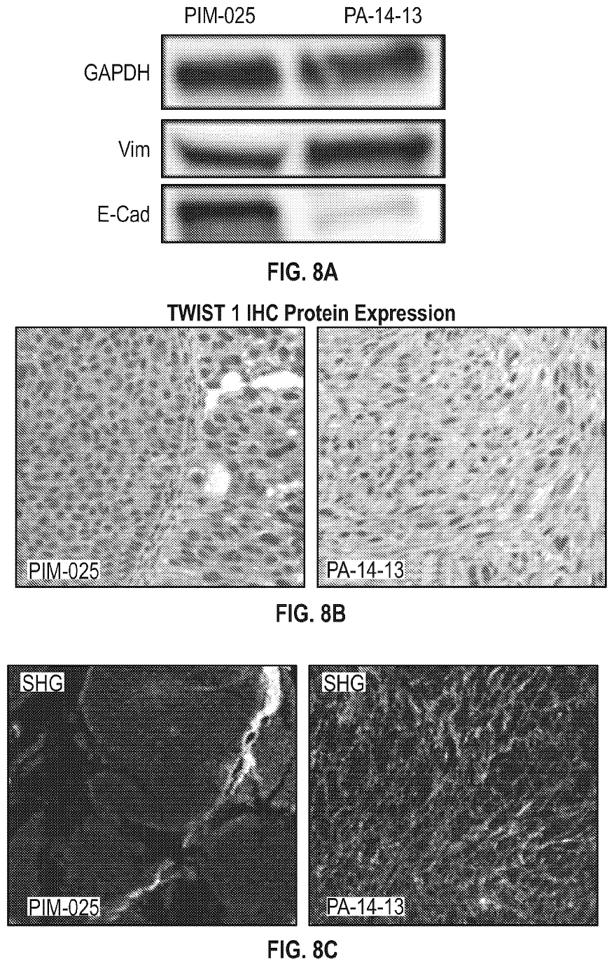

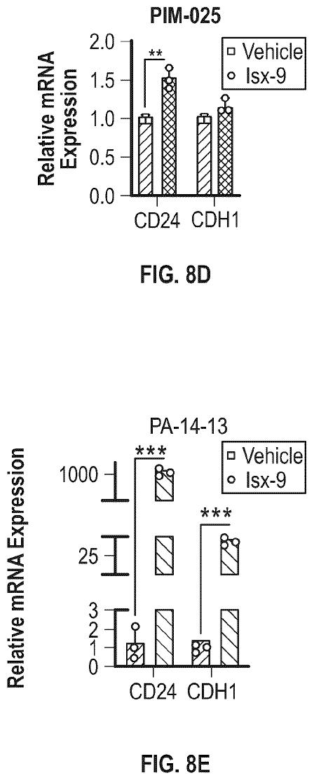

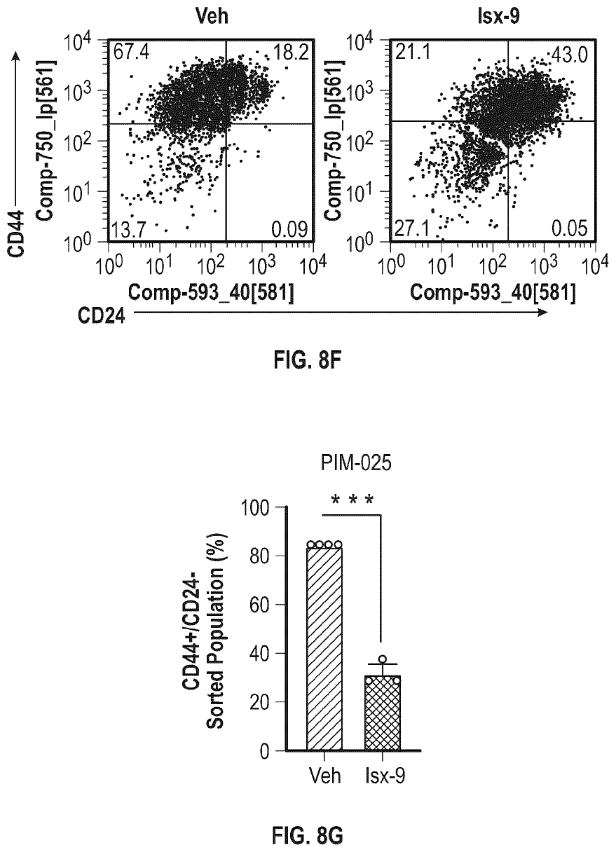

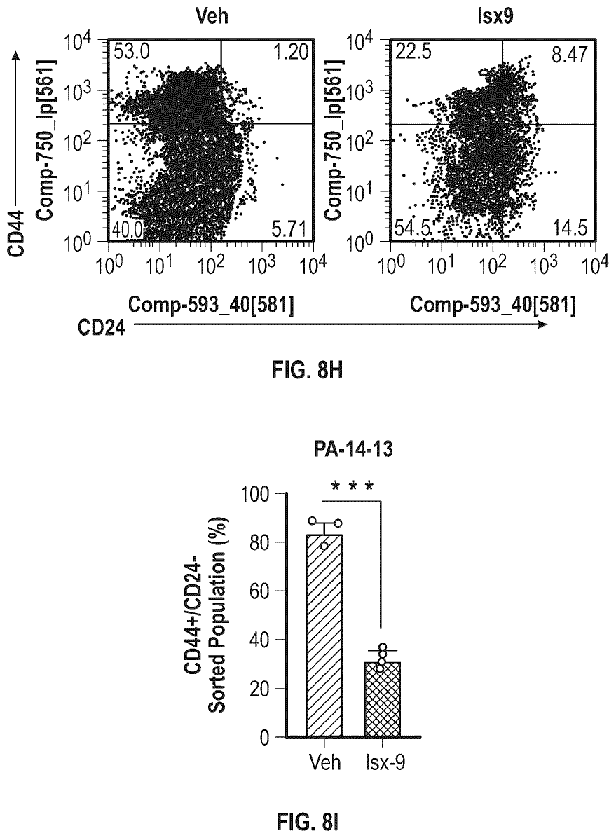

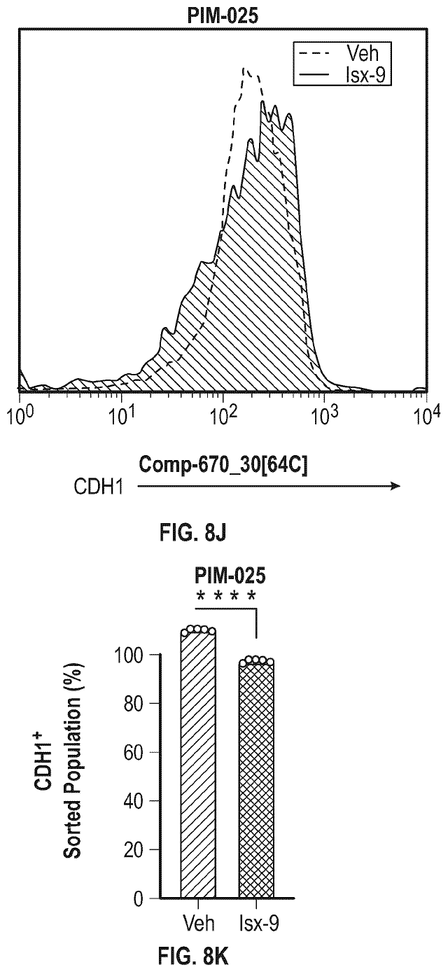

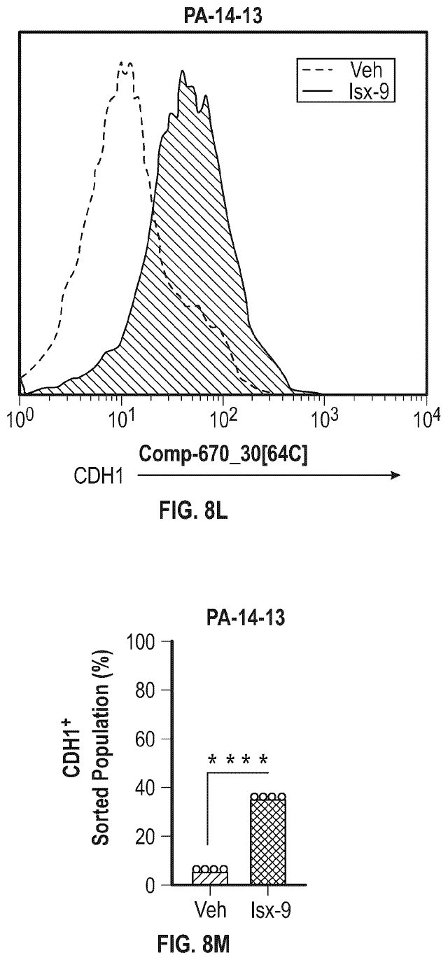

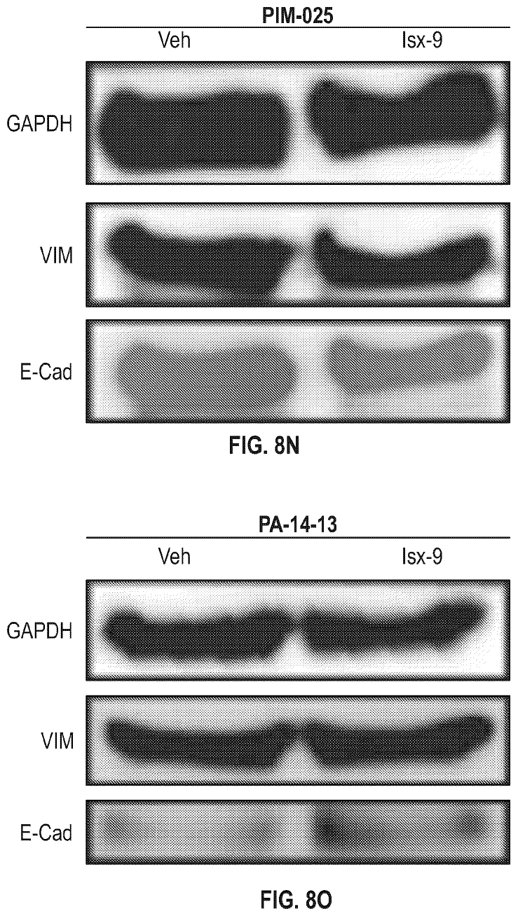

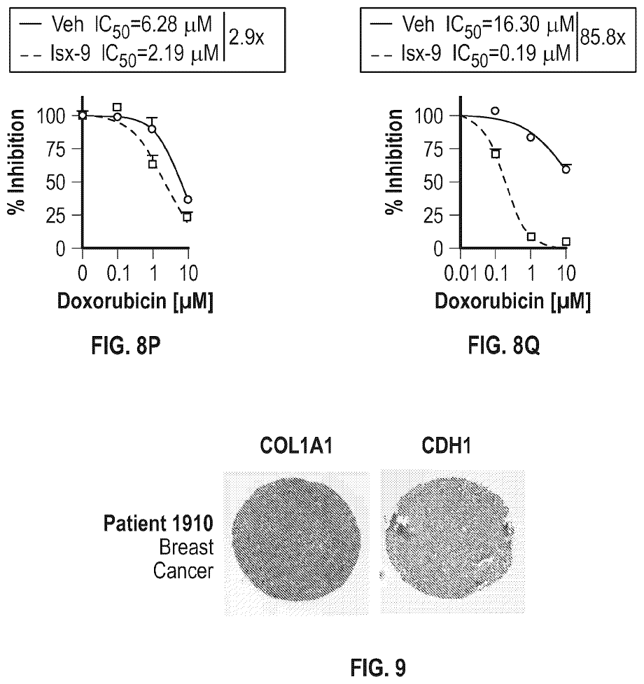

PIM-025 exhibited high co-expression of Vim and Ecad, while the PA-14-13 model exhibited strong Vim and minimal Ecad expression (FIG. 8A). FIG. 8A depicts a western blot assessment of canonical EMT markers, Vimentin and E-Cadherin, which revealed distinct expression patterns in two different patient-derived models of TNBC.

Histology profiling for the EMT transcription factor (TF) TWIST1 also revealed different localization patterns, with both nuclear and cytoplasmic localization of TWIST1 in PIM-025, but predominantly nuclear localization of TWIST1 in PA-14-13 (FIG. 8B) (see, e.g., Stemmler, M. P. et al. Nat. Cell Biol. 21, 102-112 (2019)). FIG. 8B depicts histological sections of TNBC PDX models displaying nuclear/cytoplasmic TWIST1 colocalization in PIM025 and primary nuclear TWIST1 localization in PA-14-13.

As such, these two PDX models were used to represent two of the distinct hybrid EMT phenotypes previously observed in TNBC, with the PIM-025 model displaying the characteristics of a hybrid epithelial tumor and the PA-14-13 model displaying those of a hybrid mesenchymal model. These PDX models also displayed notable differences in stromal collagen microarchitectures; the PIM-025 had sparing collagen abundance localized primarily to a tumor-stroma boundary while the PA-14-13 displayed a much more organized lamellar architecture pattern that pervaded across the tumor section (FIG. 8C). SHG imaging of FIG. 8C revealed distinct patterns of Collagen I stromal architectures in PDX sections.

Interestingly, the positive correlation between stromal abundance and a mesenchymal-like state that was observed was consistent with prior findings that increased secretion of Collagen I (Col1) by stromal cells can drive EMT (see, e.g., Yang, M.-C. et al. Am. J. Cancer Res. 4, 751-763 (2014); Olumi, A. F. et al. Cancer Res. 59, 5002-5011 (1999); and Skobe, M. et al. Proc. Natl. Acad. Sci. U.S.A 95, 1050-1055 (1998)).

As a first step towards assessing the efficacy of ISX9 to differentiate patient-derived tumor cells, PDOs from the PIM025 (“hybrid epithelial” EMT status) and PA14-13 (“hybrid mesenchymal” EMT status) TNBC PDX tumors were produced and treated with 20 μM ISX9 for 48 h. Treatment of PIM025 for 2 days induced a 1.5-fold upregulation of CD24 mRNA transcription, as well as a slight increase in CDH1 mRNA expression, which was indicative of slight CSC differentiation (FIG. 8D). PA14-13 responded even more strongly to ISX9, with a ˜1,080-fold upregulation of CD24 and ˜41-fold upregulation of CDH1 (FIG. 8E). FIG. 8D and FIG. 8E depict RT-qPCR of CD24 and CDH1 expression in PIM025 PDOs and PA-14-13 PDOs, respectively.

Using FACS analysis, confirmed was a reduction in CD44+/CD24− CSCs after 6 days of ISX9 treatment through an upregulation of CD24 in both the PIM-025 and PA-14-13 PDO lines (FIG. 8F, FIG. 8G, FIG. 8H, and FIG. 8I). These figures depict flow cytometry analysis of CD44*/CD24 CSC in (FIG. 8F and FIG. 8G) PIM025 PDOs and (FIG. 8H and FIG. 8I) PA-14-13 PDOs treated with vehicle or ISX9.

Interestingly, it was observed from FACS that only PA-14-13 cells expressed an enrichment in CDH1⁺ cells when treated with ISX9, while PIM-025 treated with ISX9 induced a slight reduction in CDH1⁺ cells (FIG. 8J, FIG. 8K, FIG. 8L, FIG. 8M). FIG. 8J and FIG. 8K depict a flow cytometry analysis of CDH1⁺ cells in PIM025 PDOs treated with vehicle or ISX9. FIG. 8L and FIG. 8M depict a flow cytometry analysis of CDH1⁺ cells in PA-14-13 PDOs treated with vehicle or ISX9.

Treated were PIM-025 and PA-14-13 cells for 6 days and observed were similar trends in E-Cadherin expression when as assessed by western blot; there were no observed differences to Vimentin expression (FIG. 8N, FIG. 8O). FIG. 8N depicts a western blot analysis of E-Cadherin and Vimentin in PIM-025 PDOs treated with vehicle or ISX9 for 6 days. FIG. 8O depicts a western blot analysis of E-Cadherin and Vimentin in PA-14-13 PDOs treated with vehicle or ISX9 for 6 days.

To determine if these effects would translate to increased sensitivity to chemotherapy, both sets of PDOs were treated with ISX9 for 5d, followed by doxorubicin for 3d. The PIM-025 cells had a 2.9-fold increase in sensitivity to doxorubicin with ISX9 pre-treatment, while the PA-14-13 cells had a much more striking 85.8-fold increase in sensitivity (FIG. 8P, FIG. 8Q). FIG. 8P and FIG. 8Q depicts results of tests showing that PIM-025 PDOs and PA-14-13 PDOs, respectively, pre-treated with ISX9 acquire increased sensitivity to doxorubicin.

These data suggested that the differing degrees of differentiation by ISX9 had a direct effect on the increase in chemo-sensitivity. It was concluded from these results that re-expression of E-Cadherin protein was a strong predictor of a ISX9-induced increase in chemo-sensitivity and also demonstrated on-target activity in two patient-derived TNBC models. Given the distinct stromal architectures and E-Cadherin expression profiles between the PIM-025 and PA-14-13 TNBC models, whether these distinct patterns could be found in additional clinical samples was identified. Histopathology of a breast cancer patient obtained from the Human Protein Atlas revealed protein expression profiles of high COL1A1 (encoding the pro-alpha1 chains of Collagen I) and low E-Cadherin that matched the profile of responsive PA-14-13 tumors (FIG. 9) (see, e.g., Thul, P. J. et al. Protein Sci. 27, 233-244 (2018)). FIG. 9 depicts abundant collagen I (COL1A1) stomal microarchitectures and low E-Cadherin (CDH1) co-expression patterns found within the same tumor of a breast cancer patient.

### Example 9—Identification of Iron-Related Biomarkers for Selection of Responsive and Non-Responsive Patients

Previous research has shown that an activated Notch1 state in T cell acute lymphoblastic lymphoma (T-ALL) may be associated with accelerated uptake of transferrin and that iron uptake may be critical for Notch1 associated disease progression. Therefore, the iron-related biomarkers of this example, discovered in TNBC, could be related to the Notch1 activation state.

Also identified were clinical biomarkers of responsive and non-responsive patients that matched directly to the invasive and non-invasive phenotypes in an embodiment of a HD Col1 model system described herein. In a previously described method, invasive and non-invasive populations out of a model system were sorted by FACS to conduct phenotypically supervised single-cell RNA sequencing data (see, e.g., Chen, K. et al. Science 24, 101991 (2021)).

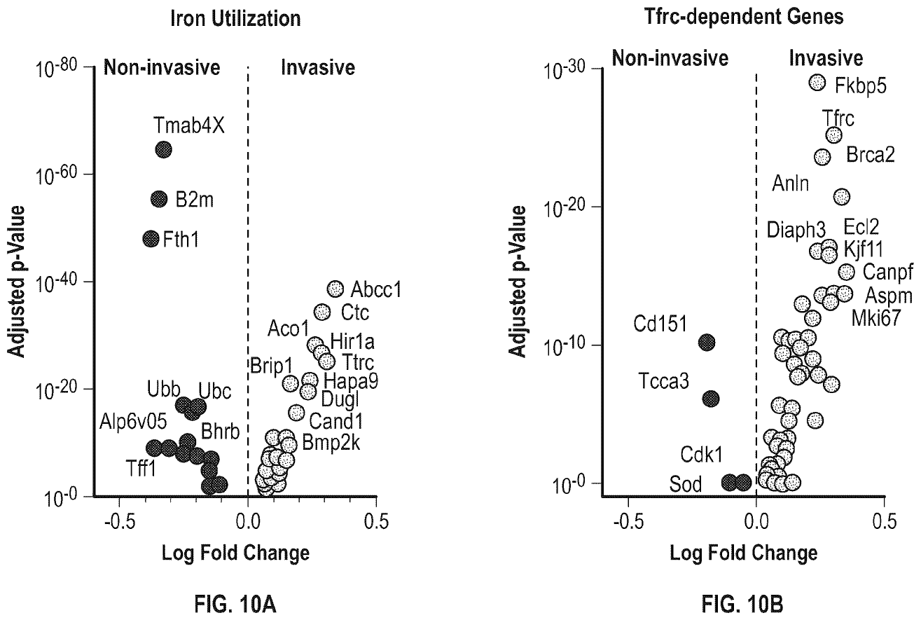

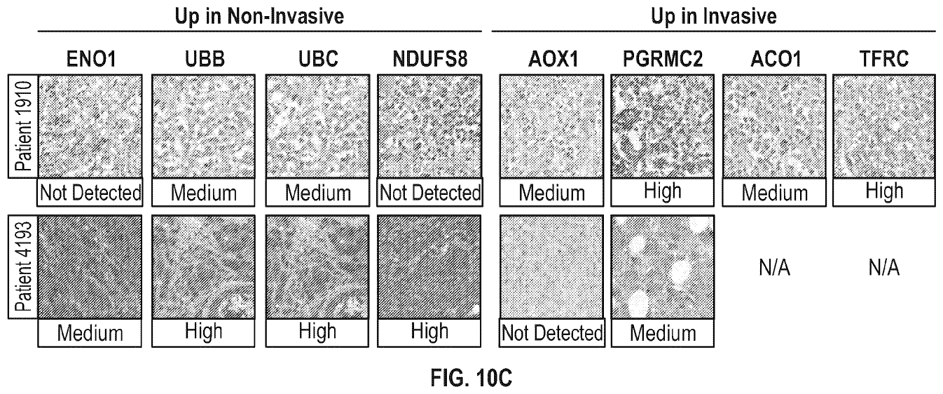

Several modules of iron-related genes were found, including iron utilization and Trfc-dependent iron transport, to be differentially expressed between invasive and non-invasive population (FIG. 10A, FIG. 10B). FIG. 10A depicts differentially expressed genes identified from PhenoSeq that are related to intracellular iron usage. FIG. 10B depicts genes whose expression decreases when Tfrc is knocked down, and are enriched in the invasive phenotype.

Histopathology of breast cancer patients obtained from the Human Protein Atlas (HPA) revealed protein expression profiles consistent with both these invasive and non-invasive gene sets. Tumor sections from Patient 1910 revealed a protein expression pattern consistent with the gene expression of the invasive phenotype, while sections from Patient 4193 were strikingly similar to the non-invasive phenotype (FIG. 10C). FIG. 10C depicts histopathology of breast cancer patients from HPA, which revealed that patient expression profiles were consistent with invasive (#1910) and non-invasive (#4193) iron-related gene sets.

The select genes with prognostic value for identifying responder v. non-responder populations included ENO1, UBB, UBC, NDUFS8, AOX1, PGRMC2, ACO1 and TFRC. Considering that patient 1910 also had profiles of high COL1A1 and low E-Cadherin, this further supported these newly identified biomarkers as predictive of Patient 1910's responsive to Isx9 and synergistic with the findings from with PDOs. Additionally, these findings provided further evidence of the significance of the 3D in vitro system to predict relevant clinical phenotypes.

### Example 10—Isoxazole Induces Differentiation Via Histone Acetylation

Inhibition of the family of histone deacetylases (HDACs) can increase acetylation, a mechanism which can be a potent suppressor of tumor growth (see, e.g., Bose, P. et al. Pharmacol. Ther. 143, 323-336 (2014)).

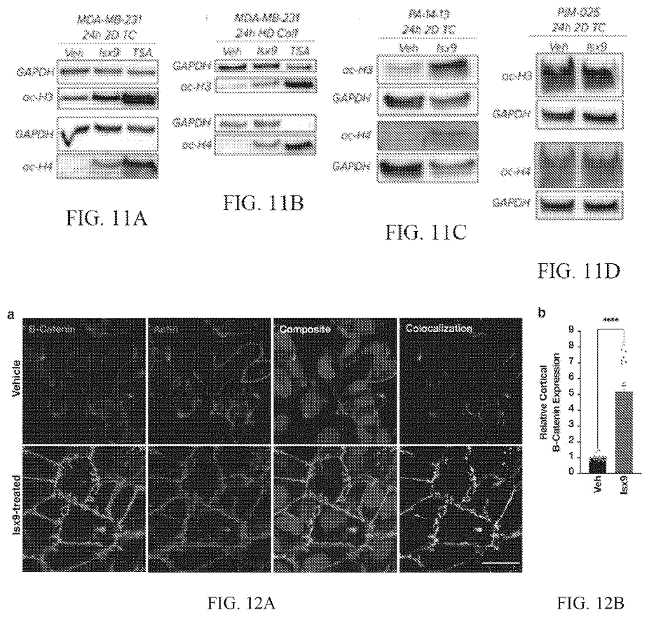

As explained in this example, it was discovered that ISX9 induces a significant increase in acetylation of histones H3 and H4, as assessed by western blot of 2D- and 3D-treated MDA-MB-231 cells for 24 hours. (FIG. 11A, FIG. 11B)

In comparison, treatment with the broad-spectrum HDAC inhibitor, TSA, similarly induced H3/H4 acetylation in both environmental conditions. Also identified was the fact that a similar increase in acetylation was found in patient-derived organoid models. Treatment of PIM025 induced a slight increase in acetylated H3/H4 expression, while PA-14-13 responded strongly, with a large increase in acetylated H3/H4 expression (FIG. 11C, FIG. 11D). Acetylation and deactylation of histones H3 and H4 are typically orchestrated by Histone Acetyltransferases (HATs), including the several HAT subfamilies, as well as HDACs (see, e.g., Marmorstein, R. et al. Cold Spring Harb. Perspect. Biol. 6, a018762 (2014)). Not wishing to be bound by any particular theory, Isx9 may bind directly to HATs and/or HDACs in order to activate histone acetylation and drive induction of MET and/or CSC differentiation.

### Example 11—Isoxazole Induces Differentiation Via Increased Localization of Beta Catenin to Cell-Cell Adhesions, Thereby Regulating Wnt Signaling

Homophilic E-Cadherin binding at tight junctions may be supported intracellularly by a set of catenins, including B-Catenin. Contact inhibition may be regulated by the interaction between E-cadherin and B-catenin, which phosphorylates Lats½ and thereby inhibits YAP nuclear entry, and proliferation (see, e.g., Kim, N. G. et al. Proc. Natl. Acad. Sci. U.S.A. 108, 11930-11935 (2011)).

As such, re-expression of B-Catenin at cell-cell junctions may equip cells with the ability to regain contact inhibition and epithelial polarity, which is lost with metastasis (see Id.).

It was discovered that treatment of MDA-MB-231 with ISX9 for 48 hours resulted in a significant increase in B-Catenin localization to the newly established cell-cell junctions of 2D-treated cells (FIG. 12A, FIG. 12B). This suggested that B-Catenin is stabilized and may regulate Wnt signaling through a distinct localization and increased abundance.

1. A method of treating a patient having cancer, the method comprising:
providing a Notch pathway activator, a RAP1 pathway activator, a RhoA pathway activator, or a combination thereof;
administering to the patient an amount of the Notch pathway activator, the RAP1 pathway activator, the RhoA pathway activator, or the combination thereof;
wherein the amount of the Notch pathway activator, the RAP1 pathway activator, the RhoA pathway activator, or the combination thereof is effective to—
(i) induce mesenchymal to epithelial transition (MET) and sensitivity to chemotherapy,
(ii) inhibit epithelial to mesenchymal transition (EMT), proliferation, maintenance, survival, and/or viability of non-CSC tumor cells, or
(iii) a combination thereof.

2. The method of claim 1, wherein the providing of the Notch pathway activator, the RAP1 pathway activator, the RhoA pathway activator, or the combination thereof comprises:
(a) providing a cell culture, wherein the cell culture is in a three-dimensional (3D) high-density collagen matrix, a patient-derived xenograph (PDX) model, or a patient-derived organoid (PDO) model, wherein the cell culture comprises a plurality of cells comprising cancer stem cells (CSCs) and non-CSC tumor cells, and wherein the plurality of cells exhibits a first expression of one or more Notch pathway genes, RAP1 pathway genes, RhoA pathway genes, or a combination thereof;
(b) contacting the cell culture and a Notch pathway activator candidate, a RAP1 pathway activator candidate, a RhoA pathway activator candidate, or a combination thereof to form a treated cell culture;
(c) determining a second expression of the one or more Notch pathway genes, RAP1 pathway genes, RhoA pathway genes, or the combination thereof exhibited by the plurality of cells of the treated cell culture;
(d) determining whether the second expression is greater than the first expression, wherein the Notch pathway activator candidate, the RAP1 pathway activator candidate, and/or the RhoA pathway activator candidate is the Notch pathway activator, the RAP1 pathway activator, and/or the RhoA pathway activator, respectively, if the second expression is greater than the first expression.

3. The method of claim 1, further comprising administering a chemotherapy, a radiation therapy, a hormone ablation therapy, a pro-apoptosis therapy, an immunotherapy, or a combination thereof to the patient prior to, concurrently with, and/or after the administering of the Notch pathway activator, the RAP1 pathway activator, the RhoA pathway activator, or the combination thereof.

4. The method of claim 1, further comprising performing surgery on the patient prior to, concurrently with, and/or after the administering of the Notch pathway activator, the RAP1 pathway activator, the RhoA pathway activator, or the combination thereof.

5. The method of claim 1, wherein the administering of the Notch pathway activator, the RAP1 pathway activator, the RhoA pathway activator, or the combination thereof comprises transiently administering the Notch pathway activator, the RAP1 pathway activator, the RhoA pathway activator, or the combination thereof by lipofection, a nanoparticle delivery system, or a nanogel delivery system.

6. The method of claim 1, wherein the cancer is a carcinoma.

7. The method of claim 1, wherein the cancer comprises one or more CSCs selected from the group consisting of a breast CSC, a fibrosarcoma CSC, a pancreatic CSC, a liver CSC, a brain CSC, a melanoma CSC, a lung CSC, a T-ALL CSC, and a prostate CSC.

8. The method of claim 1, wherein the cancer comprises one or more CSCs selected from the group consisting of CD44+/CD24−, CD133+, ALDH+, EpCAM+, CD24+, CD44+, CD90+, and CD49f+.

9. The method of claim 1, wherein the Notch pathway activator, the RAP1 pathway activator, and/or the RhoA pathway activator is a compound of formula (I), or a pharmaceutically acceptable salt thereof: 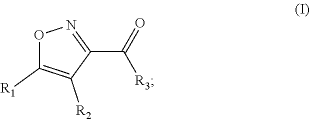
wherein R₁ is selected from the group consisting of a substituted or unsubstituted phenyl, a substituted or unsubstituted thiophenyl, and a substituent of formula (A): 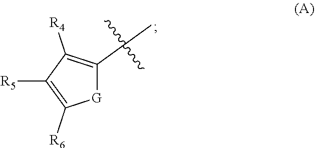
wherein R₄, R₅, and R₆, independently, are selected from the group consisting of hydrogen, hydroxy, halo, cyano, nitro, unsubstituted or substituted alkyl (C≤10), unsubstituted or substituted aryl (C≤12), unsubstituted or substituted aralkyl (C≤15), unsubstituted or substituted heteroaryl (C≤12), and unsubstituted or substituted acyl (C≤10);
wherein G is O, NH, or S;
wherein R₂ is selected from the group consisting of hydrogen, hydroxy, halo, nitro, unsubstituted or substituted alkyl (C≤10), unsubstituted or substituted alkenyl (C≤10), unsubstituted or substituted alkynyl (C≤10), unsubstituted or substituted alkoxy (C≤10), unsubstituted or substituted alkenyloxy (C≤10), unsubstituted or substituted alkynyloxy (C≤10), unsubstituted or substituted aryl (C≤12), unsubstituted or substituted aralkyl (C≤15), unsubstituted or substituted acyl (C≤10), —C(O)R₇, —OC(O)R₇, —OC(O)OR₇, —C(O)NR₈R₉, OC(O)NR₈R₉, —NR₈OR₉, and —SO₃R₇;
wherein R₇ is selected from the group consisting of hydrogen, unsubstituted or substituted alkyl (C≤10), unsubstituted or substituted aryl (C≤12), and unsubstituted or substituted aralkyl (C≤15);
wherein R₈ and R₉, independently, are selected from the group consisting of hydrogen, unsubstituted or substituted alkyl (C≤10), unsubstituted or substituted aryl (C≤12), and unsubstituted or substituted aralkyl (C≤15), or together R₈ and R₉ are unsubstituted or substituted alkanediyl (C≤6);
wherein R₃ is selected from the group consisting of unsubstituted or substituted —NH—O-alkyl (C≤10), —NHOH, —OR₁₀, and —NR₁₁R₁₂;
wherein R₁₀ is selected from the group consisting of hydrogen, substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15);
wherein R₁₁ and R₁₂, independently, are selected from the group consisting of substituted or unsubstituted alkyl (C≤10), substituted or unsubstituted alkenyl (C≤10), substituted or unsubstituted alkynyl (C≤10), substituted or unsubstituted aryl (C≤12), and substituted or unsubstituted aralkyl (C≤15), or together R₁₁ and R₁₂ form alkanediyl (C≤6), —CH₂CH₂NHCH₂CH₂—, or —CH₂CH₂OCH₂CH₂—.

10. The method of claim 9, wherein—
(i) G is S,
(ii) R₃ is —NR₁₁R₁₂, or
(iii) G is S, and R₃ is —NR₁₁R₁₂.

11. The method of claim 9, wherein the compound of formula (I) is a compound of formula (II):

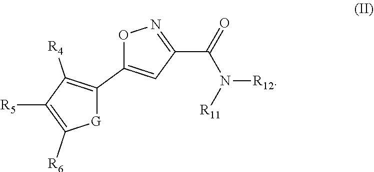

12. The method of claim 11, wherein R₁₁ or R₁₂, independently, are selected from the group consisting of cyclopropyl, cyclobutyl, cyclopentyl, and cyclohexyl, or together R₁₁ and R₁₂ form alkanediyl (C≤6), —CH₂CH₂NHCH₂CH₂—, or —CH₂CH₂OCH₂CH₂—.

13. The method of claim 1, wherein the Notch pathway activator, the RAP1 pathway activator, and/or the RhoA pathway activator is— 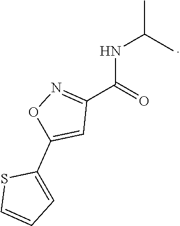 [SMILES: CC(C)NC(=O)c1cc(-c2cccs2)on1]
or a pharmaceutically acceptable salt thereof.

14. The method of claim 1, wherein the Notch pathway activator, the RAP1 pathway activator, and/or the RhoA pathway activator is— 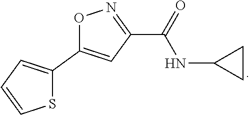 [SMILES: O=C(NC1CC1)c1cc(-c2cccs2)on1]
or a pharmaceutically acceptable salt thereof.

15. A compound of claim 9, for use in the treatment of cancer.
